SEQUENCE LISTING (16 sequences)

SEQ ID NO: 1 - DNA, 31 bp, Homo sapiens
  misc_feature (1)..(31): Primer VWC22 bind FKBP12
gcatacgtcgaattcatgggagtgcaggtgg

SEQ ID NO: 2 - DNA, 34 bp, Homo sapiens
  misc_feature (1)..(34): Primer VWC23 bind  FKBP12
gcattgctgctcgagtcattccagttttagaagc

SEQ ID NO: 3 - DNA, 39 bp, Homo sapiens
  misc_feature (1)..(39): FKBP12 D37V Primer VWC1022
gaagatggaaagaaatttgtttcctcccgggacagaaac

SEQ ID NO: 4 - DNA, 39 bp, Homo sapiens
  misc_feature (1)..(39): FKBP12 D37V Primer VWC1023
gtttctgtcccgggaggaaacaaatttctttccatcttc

SEQ ID NO: 5 - DNA, 30 bp, Homo sapiens
  misc_feature (1)..(30): FKBP12 W59L Primer VWC1364
gtgatccgaggcttggaagaaggggttgcc

SEQ ID NO: 6 - DNA, 30 bp, Homo sapiens
  misc_feature (1)..(30): FKBP12 W59L Primer VWC1365
ggcaaccccttcttccaagcctcggatcac

SEQ ID NO: 7 - DNA, 35 bp, Homo sapiens
  misc_feature (1)..(35): FKBP12 F36Y Primer VWC1366
gaagatggaaagaaatatgattcctcccgggacag

SEQ ID NO: 8 - DNA, 35 bp, Homo sapiens
  misc_feature (1)..(35): FKBP12 F36Y Primer VWC1367
ctgtcccgggaggaatcatatttctttccatcttc

SEQ ID NO: 9 - DNA, 45 bp, Homo sapiens
  misc_feature (1)..(45): FKBP12 R42Q Primer VWC1015
aaatttgattcctcccgggaccaaaacaagccctttaagtttatg

SEQ ID NO: 10 - DNA, 45 bp, Homo sapiens
  misc_feature (1)..(45): FKBP12 R42Q Primer VWC1016
cataaacttaaagggcttgttttggtcccgggaggaatcaaattt

SEQ ID NO: 11 - DNA, 39 bp, Homo sapiens
  misc_feature (1)..(39): FKBP12 H87L Primer VWC1017
gcctatggtgccactgggttgccaggcatcatcccacca

SEQ ID NO: 12 - DNA, 39 bp, Homo sapiens
  misc_feature (1)..(39): FKBP12 H87L Primer VWC1018
tggtgggatgatgcctggcaacccagtggcaccataggc

SEQ ID NO: 13 - DNA, 33 bp, Homo sapiens
  misc_feature (1)..(33): FKBP12 Y26F Primer VWC1372
acctgcgtggtgcacttcaccgggatgcttgaa

SEQ ID NO: 14 - DNA, 33 bp, Homo sapiens
  misc_feature (1)..(33): FKBP12 Y26F Primer VWC1373
ttcaagcatcccggtgaagtgcaccacgcaggt

SEQ ID NO: 15 - DNA, 18 bp, Homo sapiens
  misc_feature (1)..(18): Sequencing primer VWC422 for FKBP12
caagacccgtttagaggc

SEQ ID NO: 16 - DNA, 18 bp, Homo sapiens
  misc_feature (1)..(18): Sequencing primer VWC1053 for FKBP12
cagcctcttgctgagtgg

(19) United States
(12) Patent Application Publication
(10) Pub. No.: US 2009/0162858 A1
(43) Pub. Date: Jun. 25, 2009

(54) Orthogonal chemical inducer of dimerization

(76) Inventor: Virginia W. Cornish, New York, NY (US)

Correspondence Address: COOPER & DUNHAM, LLP, 30 Rockefeller Plaza, 20th Floor, NEW YORK, NY, 10112, US

(21) Appl. No.: 12/284,038

(22) Filed: Sep. 18, 2008

Related U.S. Application Data
(60) Provisional application No. 60/994,328, filed on Sep. 18, 2007.

(51) Int. Cl. C12Q 1/68

(52) U.S. Cl. 435/6

(57) ABSTRACT

A method for identifying a molecule as being able to bind a protein target in a cell, comprising (a) covalently bonding the molecule to trimethoprim (TMP) to form a screening molecule, (b) introducing the screening molecule into the cell which (A) expresses (i) a first fusion protein comprising a binding domain capable of binding TMP, and (ii) a second fusion protein comprising the protein target, and (B) comprises a reporter gene, wherein one of the fusion proteins comprises a DNA-binding domain and the other fusion protein comprises a transcription activation domain and wherein expression of the reporter gene is conditioned on the proximity of the DNA-binding domain to the transcription activation domain; and (c) determining whether the screening molecule binds to the first fusion protein and to the second fusion protein by determining whether the cell expresses the reporter gene, wherein expression of the reporter gene indicates that the molecule binds the protein target and wherein lack of expression of the reporter gene indicates that the molecule does not bind the protein target.

This invention has been made with government support under National Science Foundation grant CHE-9984928 and National Instititues of Health grant no. GM071754-01. Accordingly, the U.S. Government has certain rights in the invention.

Throughout this application, various publications are referenced by author or author and date. Full citations for these publications are found listed alphabetically at the end of the specification immediately preceding the claims. The disclosures of these publications in their entireties are hereby incorporated by reference into this application in order to more fully describe the state of the art as known to those skilled therein as of the date of the invention described and claimed herein.

### FIELD OF INVENTION

This invention relates to the field of identifying protein targets and their corresponding small-molecule drugs and other biomolecules using a modified yeast three hybrid system that utilizes orthogonal ligand receptor pairs.

### BACKGROUND OF THE INVENTION

The yeast three-hybrid system has been described for identifying protein targets in vivo, as well as the small molecule drugs that bind to the protein targets. (Licitra et al. (1996), represented in FIG. 2; U.S. Pat. No. 5,928,868; PCT International Publication No. WO 97/41255; Fields and Song (1989); U.S. Pat. No. 5,468,614; Yang et al. (1995); Gyuris et al. (1993); Spencer et al. (1993); Farrar et al. (1996); and Amara et al. (1997)).

In the yeast three-hybrid system, a reporter gene is constructed that is only transcribed if two proteins are brought into close proximity with one another. These two proteins are fused to an activation domain and a DNA-binding domain, respectively, to create two separate protein chimeras (also referred to as fusion proteins). When these two protein chimeras are brought into close proximity with one another they interact to form a transcriptional activator of the downstream reporter gene. In the yeast three-hybrid system, these two protein chimeras are brought into close proximity with one another by a small dimeric ligand molecule called a chemical inducer of dimerization (“CID”). A CID consists of two small molecules or “handles” covalently connected by a “linker”. If one handle of the CID binds to one of the protein chimeras and the other handle of the CID binds to the other protein chimera, then the protein chimeras are brought into close proximity since the handles are covalently connected by the linker, and the reporter gene is transcribed.

The yeast three-hybrid system is used to discover receptors for small ligands by incorporating a small ligand as one of the handles of the CID. A known protein chimera is constructed which binds to the other handle of the CID. The other protein chimera is constructed from a receptor. If this protein chimera binds to the small ligand moiety on the CID, then the reporter gene is transcribed. By repeating this process with different receptors, the yeast three-hybrid system discovers receptors which bind to a known small ligand.

The three-hybrid system is also used to screen for new ligands to known receptors by incorporating a known receptor into one of the protein chimeras. A second known protein chimera is constructed which binds to one handle of the CID. The other handle of the CID is created from a small molecule. If this other handle binds to the protein chimera constructed of a known receptor, then the reporter gene is transcribed. By repeating this process with different small molecules as the other handle of the CID, the yeast three-hybrid system screens numerous small molecules for their ability to bind to a known receptor.

A number of modifications and improvements have been made to the three-hybrid system. For example, Lin et al. (2000) improved the three-hybrid system by incorporating the known ligand-receptor pair of methotrexate (Mtx) and dihydrofolate reductase (DHFR). In this three-hybrid system, methotrexate is incorporated as a handle of the CID and DHFR is fused to a DNA binding domain or an activation domain to form one of the protein chimeras. The use of Mtx-DHFR increased the affinity of the yeast three-hybrid system to picomolar amounts. This three-hybrid screen has been incorporated into yeast and bacterial expression systems. (See PCT International Publication No. WO 01/53355 and U.S. Publication No. 2003-0203471)

In addition, several improvements have been made to the “linker” which connects the handles of the CID. “Linker” bonds may or may not contain spacer moieties and/or enzyme cleavable moieties such as phosphodiesters, glycosides, amides, esters, diesters, aldol products, or acetate moieties. The linker bond may also be a moiety providing a covalent linkage between the two-receptor binding molecules.

PCT International Publication No. WO 02/070662 A2, describes an improved “linker” with increased solubility and enhanced membrane permeability; the “linker” is a polyethylene having the general formula (CH₂—X—CH₂)ₙ, where X represents O, S, SO, or SO₂, and n is an integer from 2 to 25.

Despite these modifications made to the three-hybrid system, further improvements increases the ability to identify new ligands to known receptors or conversely to identify receptors to known ligands. Identification of the multiple ligand-receptor interactions within cells is the first step to understanding the molecular basis for these interactions.

An improved three-hybrid system has broad implications for basic biomedical research and the pharmaceutical industry. It speed-ups the research because the activity of thousands of protein variants can be measured simultaneously. It is possible to combine the three-hybrid system with existing randomization techniques to take an existing protein fold and “evolve” it into an enzyme with a new function generating useful catalysts for the pharmaceutical and chemical industries. Proteins engineered to have unique binding or catalytic properties have already proven useful as biomedical reagents, medical diagnostics, and therapeutics. Since the three-hybrid system is performed in vivo and in both prokaryotes and eukaryotes, the methodology can be applied to functional genomics and drug discovery.

### SUMMARY OF THE INVENTION

This invention provides a method for identifying a molecule as being able to bind a protein target in a cell, comprising:
(a) covalently bonding the molecule to trimethoprim (TMP) to form a screening molecule;
(b) introducing the screening molecule into the cell which (A) expresses (i) a first fusion protein comprising a binding domain capable of binding TMP, and (ii) a second fusion protein comprising the protein target, and (B) comprises a reporter gene, wherein one of the fusion proteins comprises a DNA-binding domain and the other fusion protein comprises a transcription activation domain and wherein expression of the reporter gene is conditioned on the proximity of the DNA-binding domain to the transcription activation domain; and
(c) determining whether the screening molecule binds to the first fusion protein and to the second fusion protein by determining whether the cell expresses the reporter gene,
wherein expression of the reporter gene indicates that the molecule binds the protein target and wherein lack of expression of the reporter gene indicates that the molecule does not bind the protein target.

This invention provides a method for identifying a protein target as capable of binding a molecule in a cell, comprising:
a. providing a screening molecule comprising trimethoprim (TMP) covalently bonded to the molecule;
b. introducing the screening molecule into the cell which (A) expresses (i) a first fusion protein comprising a binding domain capable of binding TMP, and (ii) a second fusion protein comprising the target protein, and (B) comprises a reporter gene, wherein one of the fusion proteins comprises a DNA-binding domain and the other fusion protein comprises a transcription activation domain and wherein expression of the reporter gene is conditioned on the proximity of the DNA-binding domain to the transcription activation domain; and
c. determining whether the protein target is capable of binding the molecule by determining whether the cell expresses the reporter gene,
wherein expression of the reporter gene indicates that the protein target is capable of binding the molecule and wherein lack of expression of the reporter gene indicates that the protein target is not capable of binding the molecule.

This invention provides a method for identifying a first molecule as a competitive inhibitor of a second molecule's binding to a protein target in a cell, comprising:
a. covalently bonding the second molecule to trimethoprim (TMP) to form a screening molecule;
b. introducing the screening molecule into the cell which (A) expresses (i) a first fusion protein comprising a binding domain capable of binding TMP, and (ii) a second fusion protein comprising the protein target, and (B) comprises a reporter gene, wherein one of the fusion proteins comprises a DNA-binding domain and the other fusion protein comprises a transcription activation domain and wherein expression of the reporter gene is conditioned on the proximity of the DNA-binding domain to the transcription activation domain;
c. quantitating cellular expression of the reporter gene;
d. introducing the first molecule into the cell repeating steps (b) and (c) in the presence of the first molecule;
wherein a decreased expression of the reporter gene in the presence of the first molecule indicates that the first molecule is a competitive inhibitor of the second molecule's binding to the protein target.

This invention provides a method for identifying a first molecule as an enhancer of a second molecule's binding to a protein target in a cell, comprising:
a. covalently bonding the second molecule to trimethoprim (TMP) to form a screening molecule;
b. introducing the screening molecule into the cell which (A) expresses (i) a first fusion protein comprising a binding domain capable of binding TMP, and (ii) a second fusion protein comprising the protein target, and (B) comprises a reporter gene, wherein one of the fusion proteins comprises a DNA-binding domain and the other fusion protein comprises a transcription activation domain and wherein expression of the reporter gene is conditioned on the proximity of the DNA-binding domain to the transcription activation domain;
c. quantitating cellular expression of the reporter gene;
d. introducing the first molecule into the cell repeating steps (b) and (c) in the presence of the first molecule;
wherein an increased expression of the reporter gene in the presence of the first molecule indicates that the first molecule is an enhancer of the second molecule's binding to the protein target.

### DESCRIPTION OF THE FIGURES

FIG. 1. Dex-Mtx protein dimerization system. A cell-permeable Dex-Mtx molecule induces dimerization of LexA-GR and DHFR-B42 protein chimeras, activating transcription of a lacZ reporter gene.

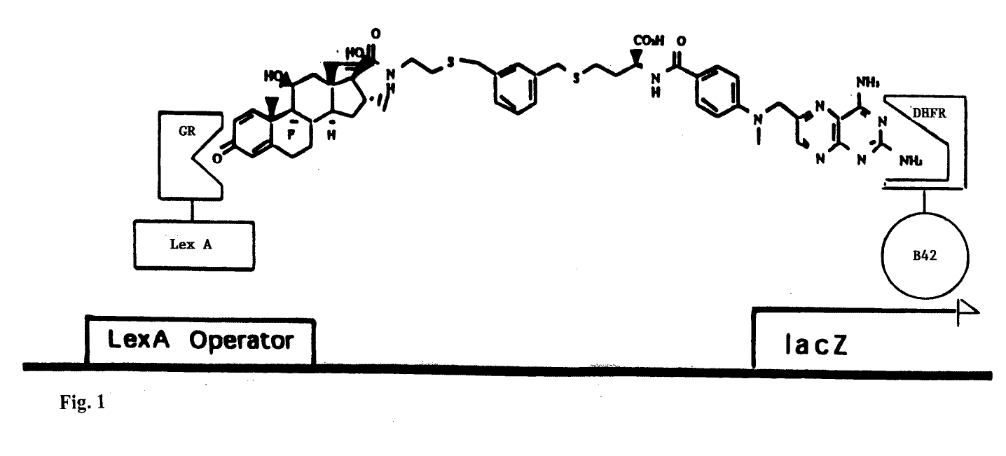

FIG. 2. DEX-MTX retrosynthesis.

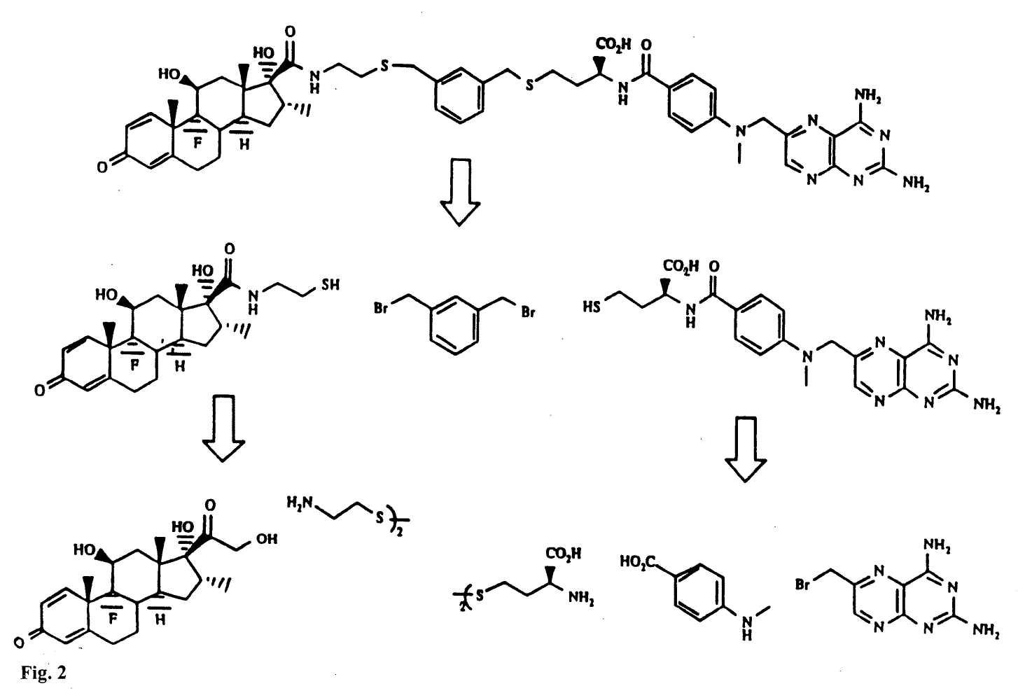

FIG. 3. Cell based assays. Yeast cells containing LexA-GR and B42-DHFR fusion proteins and the lacZ reporter gene are grown on X-gal plates with or without Dex-Mtx. Dex-Mtx dimerizes the fusion proteins, activating lacZ transcription, hydrolyzing the chromogenic substrate X-gal, and turning the cells blue. Dex-Mtx is added directly to the media in the x-gal plate. The assay takes two to five days.

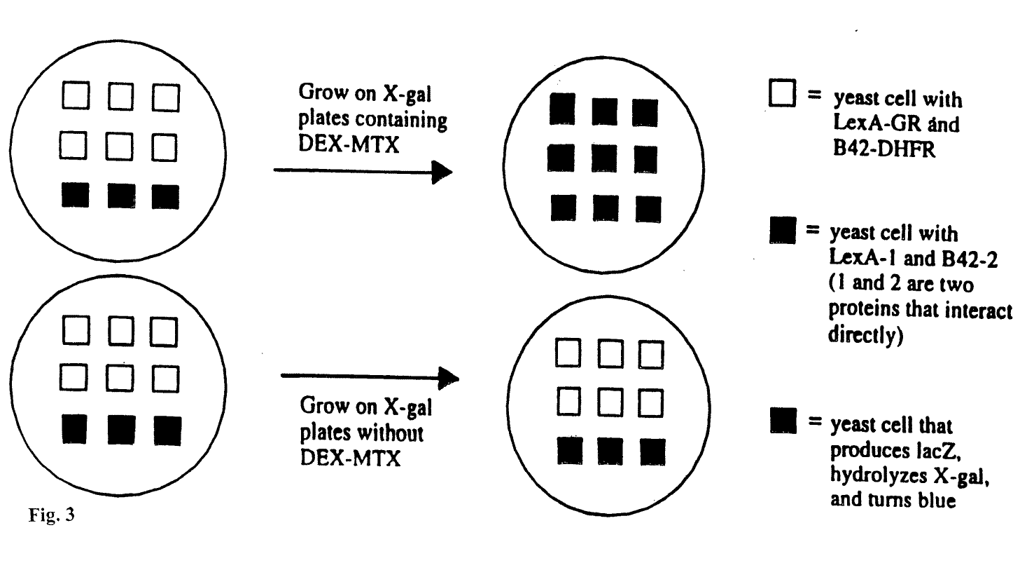

FIG. 4. Maps of the plasmids encoding the LexA-GR and B42-GR fusion proteins.

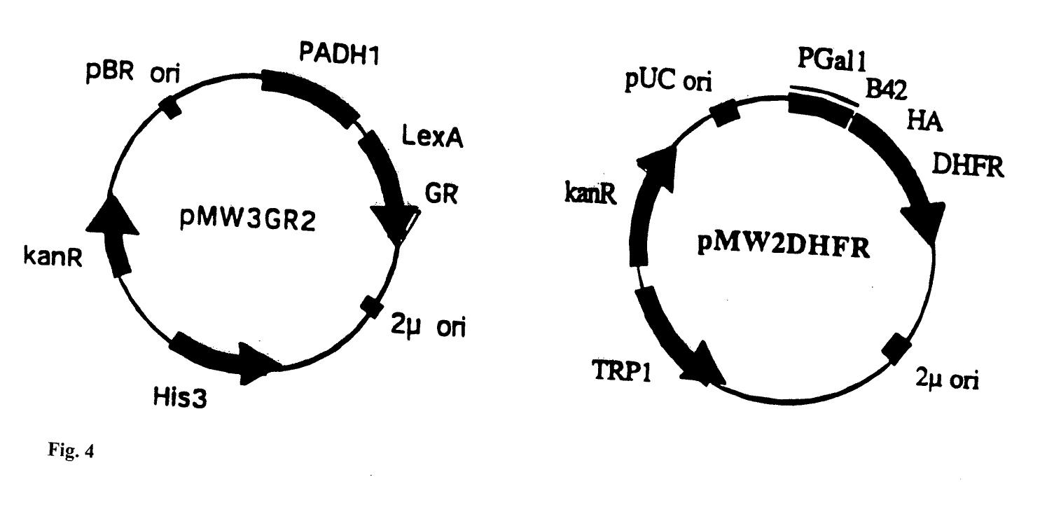

FIG. 5. Other Dex-Mtx molecules. The diamine linkers are commercially available and vary in length and hydrophobicity.

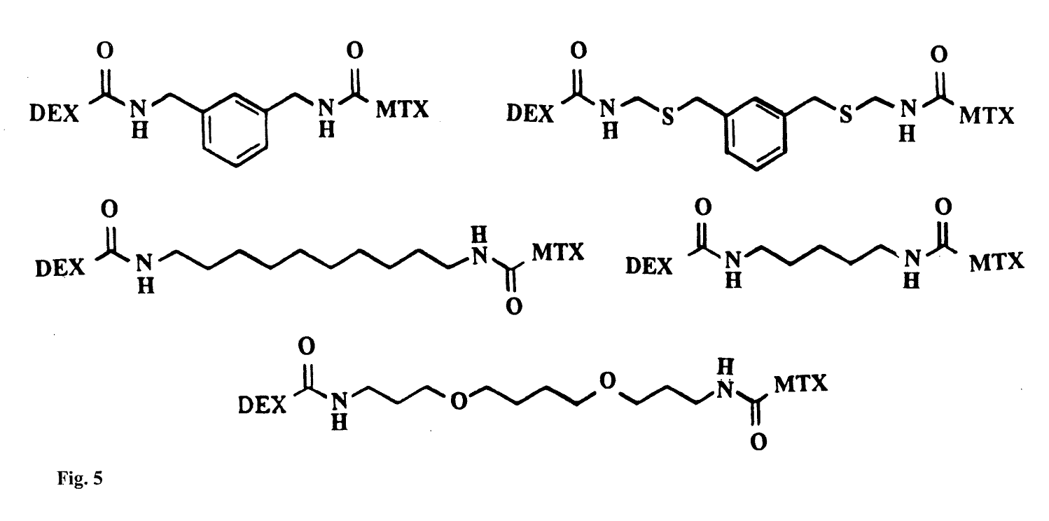

FIG. 6. Dex-cephem-Mtx retro-synthesis.

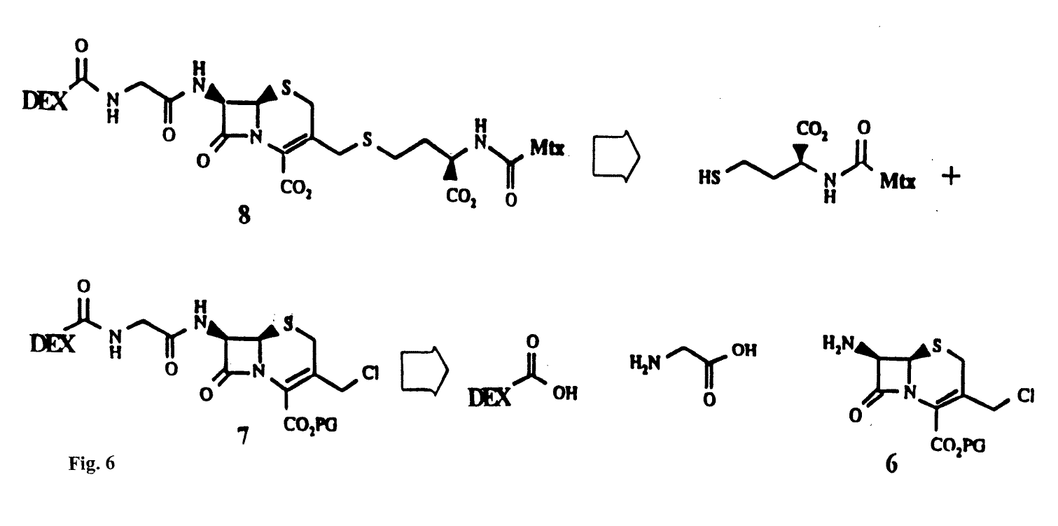

FIG. 7. X-gal plate assay of Dex-cephem-Mtx induced lacZ transcription. Yeast strains containing different LexA- and B42 chimeras, plus a lacZ reporter gene, were grown on X-gal indicator plates with or without Dex-cephem-MTX compounds: A, 1 μM Dex-MTX; B, 10 μM Dex-cephem-MTX; C, no small molecule. The strains that are dark (blue in original) even in the absence of small molecule (plate C) are positive controls on protein-protein interaction. The dark strains on plates A and B express LexA DHFR and B42-GR fusion proteins, and the white strains are negative controls, expressing only LexA and B42.

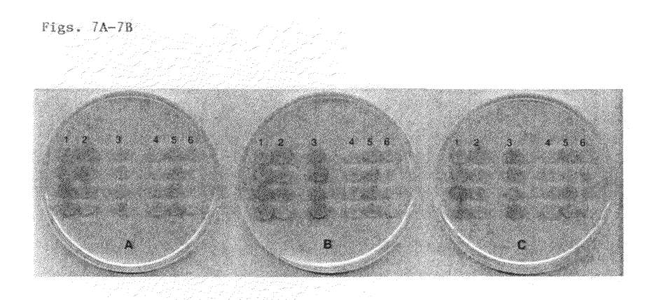

FIG. 8. The chemical handles dexamethasone (A), FK506 (B), and methotrexate (C).

FIG. 9. The structure of trimethoprim

FIG. 10. Scheme for producing a 4′-alkylamino-substituted trimethoprim.

FIG. 11. The chemical structure of the synthetic FK506 analog SLF.

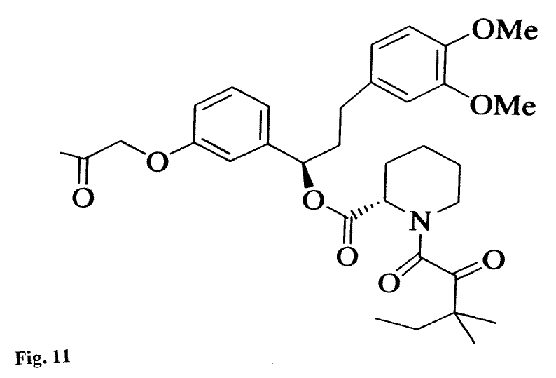

FIG. 12. A representation of yeast three-hybrid assay using Mtx-SLF. A heterodimeric ligand (Mtx-SLF) bridges a DNA-binding protein-receptor protein chimera (LexA-DHFR) and a transcription activation protein-receptor protein chimera (B42-FKBP12), effectively reconstituting a transcriptional activator and stimulating transcription of a lacZ reporter gene.

FIG. 13. The retrosynthetic analysis of trimethoprim-SLF. The carboxylic acid derivative of trimethoprim is first prepared. Trimethoprim was converted to a 4′-substituted phenol derivative by preferential cleavage of the 4′-methoxy group in hydrobromic acid. The phenol is then reacted with ethyl-5-bromovalerate in the presence of potassium tert-butoxide to yield a 4′-substituted ethyl ester. The ester is then deprotected in sodium hydroxide to yield the carboxylic acid derivative of trimethoprim. SLF acid was synthesized as described previously from L-pipecolinic acid in six steps. (Althoff et al. (2002), Amara et al. (1997), and Keenan et al. (1998)) The trimethoprim and SLF portions were then coupled to 1,10-diaminodecane in a three-component peptide coupling reaction.

FIG. 14: X-gal plate assay of trimethoprim-SLF and Mtx-SLF induced lacZ transcription from strains expressing either wild-type FKBP12 or a mutant of FKBP12. 6 separate transformants of each yeast strain were grown in the presence of 1 μM small molecule for 3 days at 30° C. All strains are derived from the parent strain V704Y (Mata, LexAeDHFR integrated into ade4 locus) possessing pMW102-FKBP12, but columns 1-8 differ in the protein that is fused to B42: 1. B42 (negative control); 2. B42-FKBP12 3. B42-FKBP12 D37V; 4. B42-FKBP12 W59L; 5. B42-FKBP12 F36Y; 6. B42-FKBP12 R42Q; 7. B42-FKBP12H97L; 8. B42-FKBP12 Y26F.

FIG. 15: Retrosynthesis of Mtx-SLF

FIG. 16: Trimethoprim-dexamethasone (TMP-Dex) yeast three-hybrid system. A heterodimeric ligand (TMP-Dex) bridges a DNA-binding domain-dihydrofolate reductase fusion protein (DBD-DHFR) on the left hand side of the figure and a transcriptional activator domain-glucocorticoid receptor fusion protein (AD-GR) on the right hand side of the figure, effectively reconstituting a transcriptional activator (DBD-AD) and activating transcription of a downstream reporter gene.

FIG. 17: LacZ transcription assays comparing the abilities of dexamethasone-trimethoprim (Dex-TMP) and dexamethasone-methotrexate (Dex-Mtx) to activate transcription in the yeast three-hybrid assay. (A) Columns I-III on each plate correspond to yeast strains containing different DNA binding domain (DBD) and/or activation domain (AD) chimeras, and a lacZ reporter gene: I, plasmid DBD-BAIT, plasmid AD-TARGET; plasmid lacZ reporter. I is a direct protein-protein interaction used as a positive control; II, integrated DBD-DHFR, plasmid AD, plasmid lacZ reporter. II has only the AD half of the AD-GR protein chimera and is used as a negative control; III, plasmid DBD-DHFR, plasmid AD-GR, plasmid lacZ reporter. III is the plasmid based yeast three-hybrid system. Plates were grown for three days with a concentration of small molecule ranging form 1-10 μM and compared to a background of no small molecule. (B) ONPG liquid assays of CID-induced lacZ transcription. The first three bars represent the data for the all plasmid system with varying concentrations of Dex-Mtx, followed by varying concentrations of Dex-TMP and no CID. The last two rows are the positive and negative controls. (C) The structures of the two small molecules used in the study Dex-Mtx and Dex-TMP.

### DETAILED DESCRIPTION OF THE INVENTION

This invention provides a method for identifying a molecule as being able to bind a protein target in a cell, comprising:
(a) covalently bonding the molecule to trimethoprim (TMP) to form a screening molecule;
(b) introducing the screening molecule into the cell which (A) expresses (i) a first fusion protein comprising a binding domain capable of binding TMP, and (ii) a second fusion protein comprising the protein target, and (B) comprises a reporter gene, wherein one of the fusion proteins comprises a DNA-binding domain and the other fusion protein comprises a transcription activation domain and wherein expression of the reporter gene is conditioned on the proximity of the DNA-binding domain to the transcription activation domain; and
(c) determining whether the screening molecule binds to the first fusion protein and to the second fusion protein by determining whether the cell expresses the reporter gene,
wherein expression of the reporter gene indicates that the molecule binds the protein target and wherein lack of expression of the reporter gene indicates that the molecule does not bind the protein target.

In an embodiment, the molecule is obtained from a combinatorial library.

This invention provides a method for identifying a protein target as capable of binding a molecule in a cell, comprising:
(a) providing a screening molecule comprising trimethoprim (TMP) covalently bonded to the molecule;
(b) introducing the screening molecule into the cell which (A) expresses (i) a first fusion protein comprising a binding domain capable of binding TMP, and (ii) a second fusion protein comprising the target protein, and (B) comprises a reporter gene, wherein one of the fusion proteins comprises a DNA-binding domain and the other fusion protein comprises a transcription activation domain and wherein expression of the reporter gene is conditioned on the proximity of the DNA-binding domain to the transcription activation domain; and
(c) determining whether the protein target is capable of binding the molecule by determining whether the cell expresses the reporter gene,
wherein expression of the reporter gene indicates that the protein target is capable of binding the molecule and wherein lack of expression of the reporter gene indicates that the protein target is not capable of binding the molecule.

In embodiments, the protein target is encoded by a DNA from the group consisting of genomic DNA, cDNA and synthetic DNA. In embodiments, the molecule has a known biological function. In embodiments, the cell is a yeast cell, a bacteria cell, an insect cell or a mammalian cell. In embodiments, the cell is S. cerevisiae or E. coli. In embodiments, the first fusion protein or the second fusion protein is dihydrofolate reductase-LexA. In embodiments, the first fusion protein or the second fusion protein is dihydrofolate reductase-B42. In embodiments, the dihydrofolate reductase in the first fusion protein or the second fusion protein is bacterial dihydrofolate reductase. In embodiments, the dihydrofolate reductase in the first fusion protein or the second fusion protein is E. coli dihydrofolate reductase. In embodiments, the reporter gene is lacZ, leu2, ura3, his3, or trp, trp1, trp2, ade2, lys2, cyh1, can1, gfp, cat, araBAD, aaDa, KanR, or Cm. In embodiments, the transcription activation domain is αNTD. In embodiments, the DNA-binding domain is λcI, AraC, LexA, Gal4, or zinc fingers.

In an embodiment of the instant methods, TMP has the following structure:

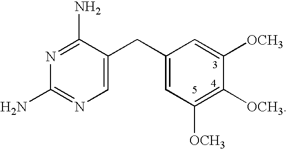

In an embodiment of the instant methods, the TMP is attached to the remainder of the screening molecule at carbon 3. In an embodiment of the instant methods, the TMP is attached to the remainder of the screening molecule at carbon 4. In an embodiment of the instant methods, the TMP is attached to the remainder of the screening molecule at carbon 5.

In embodiments of the instant methods, the TMP has the following structure:

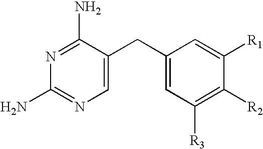

wherein if TMP is bonded to the screening molecule at R₁, then R₂ may be —H, —F, —Cl, —OH, —I, —Br, —O(CH₂)₇CH₃, —CH₂OH, —O(CH₂)₆CH₃, —OCH₂CH₂OCH₃, —CH₂O(CH₂)₃CH₃, —OCH₂CONH₂, —CH₂OCH₃, —CH₃, —O(CH₂)₃CH₃, —O(CH₂)₅CH₃, —OCH₃, —NO₂, —OSO₂CH₃, —OCH₂C₆H₅, —CF₃, —NH₂, —OCF₃, —N(CH₃)₂, —NHCOCH₃, —NHCOCH₃, —O(CH₂)₂OCH₃, and —HCH₂CH₂OCH₃; and R₃ may be —H, —OH, —CH₂OH, and —OCH₃;
wherein if TMP is bonded to the screening molecule at R₂, then R₁ may be —H, —F, —Cl, —OH, —I, —Br, —O(CH₂)₇CH₃, —CH₂OH, —O(CH₂)₆CH₃, —OCH₂CH₂OCH₃, —CH₂O(CH₂)₃CH₃, —OCH₂CONH₂, —CH₂OCH₃, —CH₃, —O(CH₂)₃CH₃, —O(CH₂)₅CH₃, —OCH₃, —NO₂, —OSO₂CH₃, —OCH₂C₆H₅, —CF₃, —NH₂, —OCF₃, —N(CH₃)₂, —NHCOCH₃, —NHCOCH₃, —O(CH₂)₂OCH₃, and —HCH₂CH₂OCH₃; and R₃ may be —H, —OH, —CH₂OH, and —OCH₃;
wherein if TMP is bonded to the screening molecule at R₃, then R₁ may be —H, —F, —Cl, —OH, —I, —Br, —O(CH₂)₇CH₃, —CH₂OH, —O(CH₂)₆CH₃, —OCH₂CH₂OCH₃, —CH₂O(CH₂)₃CH₃, —OCH₂CONH₂, —CH₂OCH₃, —CH₃, —O(CH₂)₃CH₃, —O(CH₂)₅CH₃, —OCH₃, —NO₂, —OSO₂CH₃, —OCH₂C₆H₅, —CF₃, —NH₂, —OCF₃, —N(CH₃)₂, —NHCOCH₃, —NHCOCH₃, —O(CH₂)₂OCH₃, and —HCH₂CH₂OCH₃; and R₂ may be —H, —F, —Cl, —OH, —I, —Br, —O(CH₂)₇CH₃, —CH₂OH, —O(CH₂)₆CH₃, —OCH₂CH₂OCH₃, —CH₂O(CH₂)₃CH₃, —OCH₂CONH₂, —CH₂OCH₃, —CH₃, —O(CH₂)₃CH₃, —O(CH₂)₅CH₃, —OCH₃, —NO₂, —OSO₂CH₃, —OCH₂C₆H₅, —CF₃, —NH₂, —OCF₃, —N(CH₃)₂, —NHCOCH₃, —NHCOCH₃, —O(CH₂)₂OCH₃, and —HCH₂CH₂OCH₃.

This invention provides the instant methods wherein the screening molecule has the formula:

TMP-X—B—Y—H2
wherein H2 is the molecule; and
wherein each of X and Y and B is a covalently linked spacer, which can be present or absent and, if present, each may be the same or different.

In an embodiment, in the screening molecule X and Y are, independently, a polyethylene linker having the general formula (CH₂-Z-CH₂)ₙ, where Z represents O, S, SO, or SO₂, and n is an integer from 2 to 25.

In an embodiment, X and Y are, independently, a polyethylene linker where Z is O. In an embodiment, X and Y are, independently, a polyethylene linker having the general formula (CH₂-Z-CH₂)ₙ, where n is an integer from 2 to 5. In an embodiment, X and Y are have different molecular structures.

In an embodiment, in the screening molecule B is present and is an enzyme cleavable moiety capable of binding to the enzyme with an IC₅₀ of less than 100 mM. In embodiments B is capable of binding to the enzyme with an IC₅₀ of less than 10 mM, or B is capable of binding to the enzyme with an IC₅₀ of less than 1 mM.

In an embodiment, B is cleavable by a transferase, hydrolase, lyase, isomerase, or ligase. In an embodiment, the transferase is a carbon transferase, an aldehyde or ketone transferase, an acyl transferase, a glycosyl transferase, an alkyl or aryl trasferase, a N-containing group transferase, a P-containing group transferase, an S-containing group transferase, an O-containing group transferase, or a Se-containing group transferase. In an embodiment, the hydrolase is an ester hydrolase, a glycosidic hydrolase, an ether hydrolase, a peptide hydrolase, a C—N (non-peptide) hydrolase, an acid anhydride hydrolase, a C—C hydrolase, a P—N hydrolase, a S—N hydrolase, a C—P hydrolase, a C—O hydrolase (non-ester, non-ether), or a S—S hydrolase. In an embodiment, the lyase is a C—C lyase, a C—O lyase, a C—N lyase, a C—S lyase, or a P—O lyase. In an embodiment, the isomerase is a racemase, epimerase, cis-trans isomerase, intra-oxidoreductase, intra-transferase (mutase), or intramolecular lyase. In an embodiment, the ligase is a C—O ligase, a C—S ligase, a C—N ligase, a C—C ligase, or a P—O ligase. In an embodiment, B is a phosphodiester, glycoside, amide, ester, diester, cephem or an aldol product moiety.

In an embodiment, the H2 is derived from a compound selected from the group consisting of lipids, halides, alcohols, aldehydes, alkanes, alkenes, alkynes, alkyls, alkaloids, amines, aromatic hydrocarbons, esters, ethers, phenols, nitriles, anhydrides, amides, imines, enamines, aldols, organometallics, amine oxide, cyanohydrin, organocadmium, quarternary ammonium salts, carboxylic acid anhydrides, aryl halids, carboxylate acids, nucleic acids, polypeptides, steroids, hormones, nuclear receptor ligands, cofactors, antibiotics, sugars, enzyme inhibitors, drugs, or a derivative thereof.

In an embodiment, H2 is dexamethasone, 3,5,3′-triiodothyronine, trans-retinoic acid, biotin, coumermycin, tetracycline, lactose, methotrexate, FK506, FK506 analogs, cephem, testosterone, estrogen, progesterone, cortisone, cyclosporin, cannabinoid, rapamycin, maltose, nickel, 2,4-diaminopteridine, novobiocin, glutathione, trimethoprim, trimethoprim analogs, or a derivative thereof.

In an embodiment, the determination of whether the cell expresses the reporter gene is performed by Fluorescence Activated Cell Sorting (FACS), or observation of the activity of a gene transcription marker.

In an embodiment, the gene transcription marker is a Green Fluorescence Protein, LacZ-β-galactosidases, luciferase, antibiotic resistant β-lactamases, yeast markers, TetR (tetracycline resistance), KanR (kanamycin resistance), Cm (chloroamphenicol resistance), aada (spectinomycin resistance), araBAD, URA3, or PLV.

This invention provides a method for identifying a first molecule as a competitive inhibitor of a second molecule's binding to a protein target in a cell, comprising:
(a) covalently bonding the second molecule to trimethoprim (TMP) to form a screening molecule;
(b) introducing the screening molecule into the cell which (A) expresses (i) a first fusion protein comprising a binding domain capable of binding TMP, and (ii) a second fusion protein comprising the protein target, and (B) comprises a reporter gene, wherein one of the fusion proteins comprises a DNA-binding domain and the other fusion protein comprises a transcription activation domain and wherein expression of the reporter gene is conditioned on the proximity of the DNA-binding domain to the transcription activation domain;
(c) quantitating cellular expression of the reporter gene;
(d) introducing the first molecule into the cell repeating steps (b) and (c) in the presence of the first molecule;
wherein a decreased expression of the reporter gene in the presence of the first molecule indicates that the first molecule is a competitive inhibitor of the second molecule's binding to the protein target.

This invention provides a method for identifying a first molecule as an enhancer of a second molecule's binding to a protein target in a cell, comprising:
(a) covalently bonding the second molecule to trimethoprim (TMP) to form a screening molecule;
(b) introducing the screening molecule into the cell which (A) expresses (i) a first fusion protein comprising a binding domain capable of binding TMP, and (ii) a second fusion protein comprising the protein target, and (B) comprises a reporter gene, wherein one of the fusion proteins comprises a DNA-binding domain and the other fusion protein comprises a transcription activation domain and wherein expression of the reporter gene is conditioned on the proximity of the DNA-binding domain to the transcription activation domain;
(c) quantitating cellular expression of the reporter gene;
(d) introducing the first molecule into the cell repeating steps (b) and (c) in the presence of the first molecule;
wherein an increased expression of the reporter gene in the presence of the first molecule indicates that the first molecule is an enhancer of the second molecule's binding to the protein target.

In an embodiment, the cell is a yeast cell, a bacteria cell, an insect cell or a mammalian cell. In an embodiment, the cell is S. cerevisiae or E. coli. In an embodiment, the first fusion protein or the second fusion protein is dihydrofolate reductase-LexA. In an embodiment, the first fusion protein or the second fusion protein is dihydrofolate reductase-B42. In an embodiment, the dihydrofolate reductase in the first fusion protein or the second fusion protein is bacterial dihydrofolate. In an embodiment, the dihydrofolate in the first fusion protein or the second fusion protein is E. coli dihydrofolate. In embodiments, the reporter gene is lacZ, leu2, ura3, his3, or trp, trp1, trp2, ade2, lys2, cyh1, can1, gfp, cat, araBAD, aaDa, KanR, or Cm. In an embodiment, the transcription activation domain is αNTD. In embodiments, the DNA-binding domain is λcI, AraC, LexA, Gal4, or zinc fingers.

In embodiments, the TMP has the following structure:

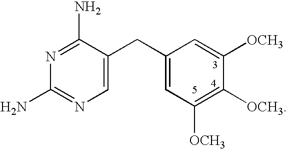

In an embodiment, the TMP is attached to the remainder of the screening molecule at carbon 3. In an embodiment, the TMP is attached to the remainder of the screening molecule at carbon 4. In an embodiment, the TMP is attached to the remainder of the screening molecule at carbon 5. In embodiments, the TMP has the following structure:

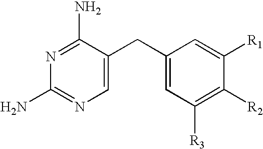

wherein if TMP is bonded to the screening molecule at R₁, then R₂ may be —H, —F, —Cl, —OH, —I, —Br, —O(CH₂)₇CH₃, —CH₂OH, —O(CH₂)₆CH₃, —OCH₂CH₂OCH₃, —CH₂O(CH₂)₃CH₃, —OCH₂CONH₂, —CH₂OCH₃, —CH₃, —O(CH₂)₃CH₃, —O(CH₂)₅CH₃, —OCH₃, —NO₂, —OSO₂CH₃, —OCH₂C₆H₅, —CF₃, —NH₂, —OCF₃, —N(CH₃)₂, —NHCOCH₃, —NHCOCH₃, —O(CH₂)₂OCH₃, and —HCH₂CH₂OCH₃; and R₃ may be —H, —OH, —CH₂OH, and —OCH₃;
wherein if TMP is bonded to the screening molecule at R₂, then R₁ may be —H, —F, —Cl, —OH, —I, —Br, —O(CH₂)₇CH₃, —CH₂OH, —O(CH₂)₆CH₃, —OCH₂CH₂OCH₃, —CH₂O(CH₂)₃CH₃, —OCH₂CONH₂, —CH₂OCH₃, —CH₃, —O(CH₂)₃CH₃, —O(CH₂)₅CH₃, —OCH₃, —NO₂, —OSO₂CH₃, —OCH₂C₆H₅, —CF₃, —NH₂, —OCF₃, —N(CH₃)₂, —NHCOCH₃, —NHCOCH₃, —O(CH₂)₂OCH₃, and —HCH₂CH₂OCH₃; and R₃ may be —H, —OH, —CH₂OH, and —OCH₃;
wherein if TMP is bonded to the screening molecule at R₃, then R₁ may be —H, —F, —Cl, —OH, —I, —Br, —O(CH₂)₇CH₃, —CH₂OH, —O(CH₂)₆CH₃, —OCH₂CH₂OCH₃, —CH₂O(CH₂)₃CH₃, —OCH₂CONH₂, —CH₂OCH₃, —CH₃, —O(CH₂)₃CH₃, —O(CH₂)₅CH₃, —OCH₃, —NO₂, —OSO₂CH₃, —OCH₂C₆H₅, —CF₃, —NH₂, —OCF₃, —N(CH₃)₂, —NHCOCH₃, —NHCOCH₃, —O(CH₂)₂OCH₃, and —HCH₂CH₂OCH₃; and R₂ may be —H, —F, —Cl, —OH, —I, —Br, —O(CH₂)₇CH₃, —CH₂OH, —O(CH₂)₆CH₃, —OCH₂CH₂OCH₃, —CH₂O(CH₂)₃CH₃, —OCH₂CONH₂, —CH₂OCH₃, —CH₃, —O(CH₂)₃CH₃, —O(CH₂)₅CH₃, —OCH₃, —NO₂, —OSO₂CH₃, —OCH₂C₆H₅, —CF₃, —NH₂, —OCF₃, —N(CH₃)₂, —NHCOCH₃, —NHCOCH₃, —O(CH₂)₂OCH₃, and —HCH₂CH₂OCH₃.

### EXPERIMENTAL DETAILS

### Example 1

A Y3H system whose components are orthogonal to the yeast cellular machinery is more sensitive than current systems because the chemical inducer of dimerization (CID) is not buffered by endogenous receptors. The use of an orthogonal ligand receptor pair in a mammalian three-hybrid system increases the sensitivity of identifying ligand interactions of mammalian receptors within their natural environment. Furthermore, in using the Y3H to study protein-protein interactions of biological importance, sensitivity to the small molecule is necessary. For example, the Y3H system can be engineered to dimerize two components of a cell signaling pathway upon addition of the CID. Control of the interaction is afforded by careful control of the CID cellular concentration. By limiting the cellular CID binding targets to only the chimeric receptors, control of concentration is improved.

### Components of the Yeast Three-Hybrid System

The yeast three-hybrid system consists of several components including: chemical inducers of dimerization or CIDs which consist of two small molecule handles connected by a linker; protein chimeras which consist of separate proteins fused to either an activation domain or a DNA-binding domain; a reporter gene which is only transcribed if these two protein chimeras are brought into close proximity with one another; and a host cell. The following section provides greater detail on these components.

### I. Handles of Chemical Inducers of Dimerization

Several small molecules or “chemical inducers of dimerization” have previously been reported which can bridge protein dimerization. (Spencer et al. (1996); Spencer et al. (1995); Spencer et al. (1993); and Crabtree et al. (1996)).

There are many types of small molecule ligands described in the art that can be incorporated as handles of the CID for the yeast three-hybrid system. See, for example, Licitra (1996); U.S. Pat. No. 5,928,868; Crabtree et al. (1996); PCT International Publication No. WO 94/18317; PCT International Publication No. WO 96/13613; PCT International Publication No. Wo 96/06097; PCT International Publication No. WO 97/41255; Bergmann et al. (1994); Lin et al. (2000); PCT International Publication No. WO 02/070662; PCT International Publication No. WO 01/53355; and U.S. Publication No. 2003-0203471).

Ideally, a handle binds its receptor with high affinity (≦100 nM), crosses cell membranes yet is inert to modification or degradation, is available in reasonable quantities, and presents a convenient side-chain for routine chemical derivatization that does not disrupt receptor binding.

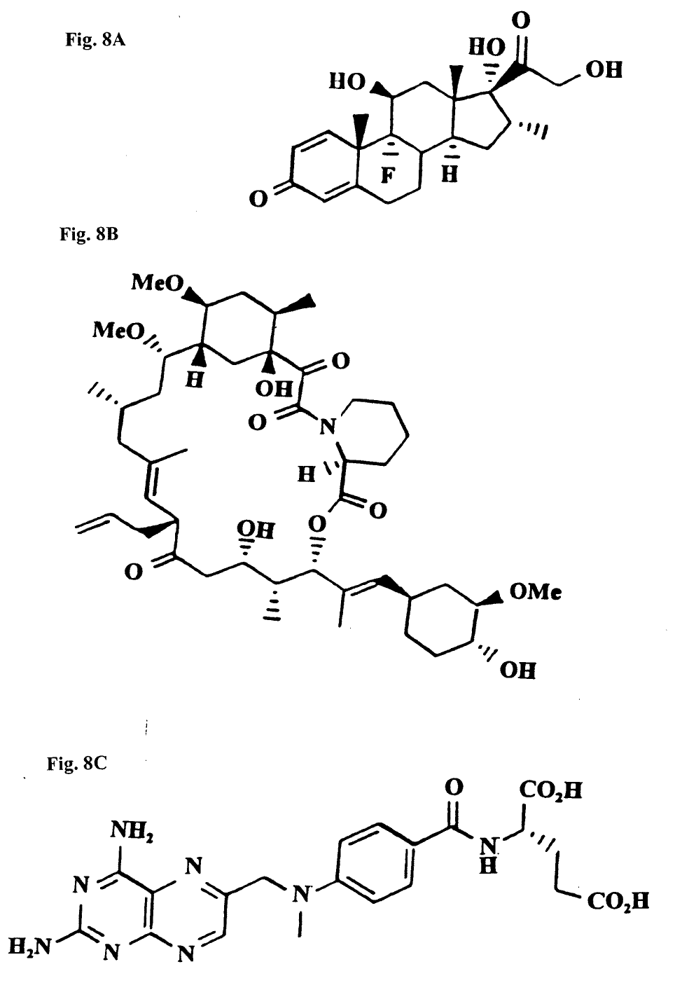

Dexamethasone (DEX) is one example of a handle used in the prior art. Dexamethasone (DEX) (FIG. 8A) binds rat glucocorticoid receptor (GR), (Chakraborti et al. (1991)) regulates the in vivo activity and nuclear localization of GR fusion proteins (Picard et al. (1987)), and is commercially available. Affinity columns for rGR have been prepared via the C₂₀ α-hydroxy ketone of dexamethasone. (Govindan et al. (1980) and Manz et al. (1983)).

Methotrexate (MTX) has also been used as a handle in place of FK506 since FK506 is not an ideal handle (See Lin et al. (2000); Spencer et al. (1993); Spencer et al. (1995); Spencer et al. (1996); Pruschy et al. (1994); Wagner et al. (1995); Wagner et al. (1998); and Coleman et al. (1989)) MTX is commercially available and can be modified selectively at its γ-carboxylate without disrupting dihydrofolate reductase (DHFR) binding. (Kralovec et al. (1989); and Bolin et al. (1982))

Other handles include, for example, steroids; enzyme inhibitors; drugs; hormones, such as the thyroid hormone 3,5,3′-triiodothyronine; ligands for nuclear receptors, such as be retinoic acids; general cofactors, such as Biotin; and antibiotics.

Disclosed herein is an improvement over the small molecule ligands described in the prior art by incorporating trimethoprim or an analog thereof trimethoprim (TMP) as one of the handles of the CID. While TMP is only one of a number of molecules having the characteristics appropriate for use as a handle, TMP has been found to be an exceptional handle for a CID.

### I (a) Trimethoprim and Analogs as a CID Handle

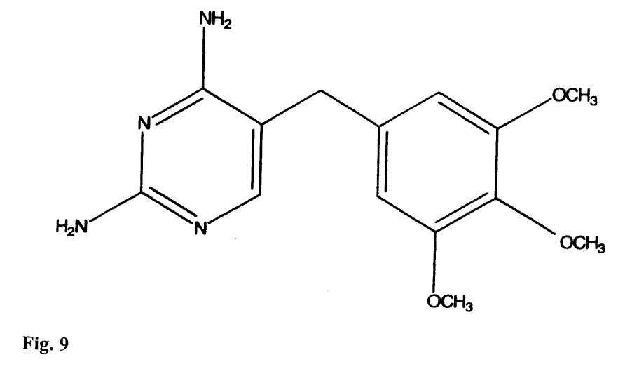

The structure of trimethoprim or 2,4-diamino-5-(3,4,5-trimethoxybenzyl)pyrimidine is shown in FIG. 9. For the synthesis of trimethoprim see U.S. Pat. No. 3,049,544, U.S. Pat. No. 3,341,541, Roth et al. (1980), and Roth et al. (1981).

Trimethopr(DHFR). Trimethoprim has a high affinity for E. coli DHFR (“eDHFR”), but a low affinity for the mammalian form of DHFR. (Baccanari et al.) Trimethoprim derivatives have previously been used to label eDHFR fusion proteins in mammalian cells with little or no background noise resulting from binding to endogenous DHFR. (Miller et al. (2005)) Trimethoprim is easily derivatized without disrupting its binding to eDHFR. (Roth et al. (1981))

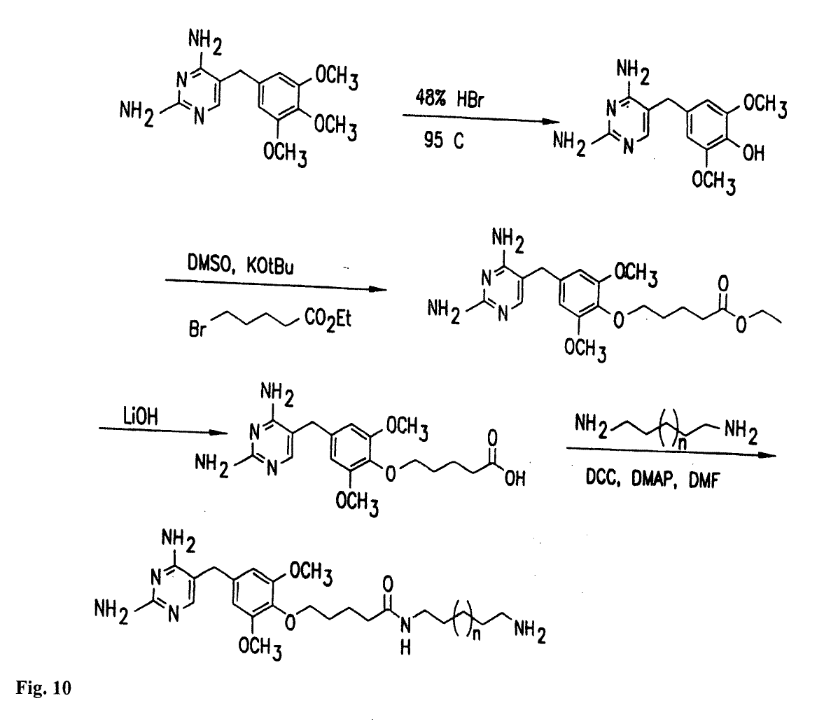

In an embodiment of the invention, labeled trimethoprim is prepared which is substituted at the 4′ position. 4′-substituted trimethoprim retains nanomolar affinity for E. coli DHFR as well as selectivity over mammalian forms of DHFR if the substituent is attached via an alkyl linker with a chain length longer than 3 carbons (Roth et al. (1981); and Kuyper et al. (1985)). Trimethoprim is selectively demethylated at the 4′ position according to the method of Brossi et al. (1971). The resulting phenol compound is alkylated via reaction with a bromo-alkanoate ester (Kuyper et al. (1985)). The ester is then hydrolyzed and reacted with a diamino alkane of desired length using a carbodiimide, or other peptide coupling reagent to form the product, a 4′-alkylamino-substituted trimethoprim (see FIG. 10.)

Additional analogs of trimethoprim have been disclosed in the art which may be used as a handle of the CID in the three-hybrid system. (See for example, King et al. (1992); Wust and Schwarzenbach (1983); Walzer et al. (1993); Then et al. (1982); and Iversen et al. (1984)). Examples of trimethoprim analogs include the following structures:

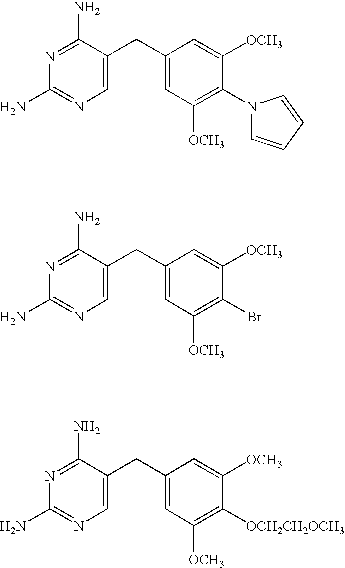

In addition, TMP may have the following structure:

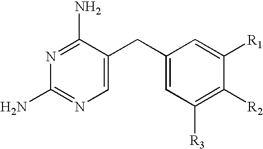

wherein if TMP is bonded to the screening molecule at R₁, then R₂ may be —H, —F, —Cl, —OH, —I, —Br, —O(CH₂)₇CH₃, —CH₂OH, —O(CH₂)₆CH₃, —OCH₂CH₂OCH₃, —CH₂O(CH₂)₃CH₃, —OCH₂CONH₂, —CH₂OCH₃, —CH₃, —O(CH₂)₃CH₃, —O(CH₂)₅CH₃, —OCH₃, —NO₂, —OSO₂CH₃, —OCH₂C₆H₅, —CF₃, —NH₂OCF₃, —N(CH₃)₂, —NHCOCH₃, —NHCOCH₃, —O(CH₂)₂OCH₃, and —HCH₂CH₂OCH₃; and R₃ may be —H, —OH, —CH₂OH, and —OCH₃;
wherein if TMP is bonded to the screening molecule at R₂, then R₁ may be —H, —F, —Cl, —OH, —I, —Br, —O(CH₂)₇CH₃, —CH₂OH, —O(CH₂)₆CH₃, —OCH₂CH₂OCH₃, —CH₂O(CH₂)₃CH₃, —OCH₂CONH₂, —CH₂OCH₃, —CH₃, —O(CH₂)₃CH₃, —O(CH₂)₅CH₃, —OCH₃, —NO₂, —OSO₂CH₃, —OCH₂C₆H₅, —CF₃, —NH₂, —OCF₃, —N(CH₃)₂, —NHCOCH₃, —NHCOCH₃, —O(CH₂)₂OCH₃, and —HCH₂CH₂OCH₃; and R₃ may be —H, —OH, —CH₂OH, and —OCH₃;
wherein if TMP is bonded to the screening molecule at R₃, then R₁ may be —H, —F, —Cl, —OH, —I, —Br, —O(CH₂)₇CH₃, —CH₂OH, —O(CH₂)₆CH₃, —OCH₂CH₂OCH₃, —CH₂O(CH₂)₃CH₃, —OCH₂CONH₂, —CH₂OCH₃, —CH₃, —O(CH₂)₃CH₃, —O(CH₂)₅CH₃, —OCH₃, —NO₂, —OSO₂CH₃, —OCH₂C₆H₅, —CF₃, —NH₂, —OCF₃, —N(CH₃)₂, —NHCOCH₃, —NHCOCH₃, —O(CH₂)₂OCH₃, and —HCH₂CH₂OCH₃; and R₂ may be —H, —F, —Cl, —OH, —I, —Br, —O(CH₂)₇CH₃, —CH₂OH, —O(CH₂)₆CH₃, —OCH₂CH₂OCH₃, —CH₂O(CH₂)₃CH₃, —OCH₂CONH₂, —CH₂OCH₃, —CH₃, —O(CH₂)₃CH₃, —O(CH₂)₅CH₃, —OCH₃, —NO₂, —OSO₂CH₃OCH₂C₆H₅, —CF₃, —NH₂, —OCF₃, —N(CH₃)₂, —NHCOCH₃, —NHCOCH₃, —O(CH₂)₂OCH₃, and —HCH₂CH₂OCH₃.

By incorporating the trimethoprim moiety or one of its analogs, collectively referred to as “TMP”, as one of the handles of the CID in a three-hybrid (Y3H) system, it allows the TMP handle to bind with high affinity to bacterial forms of DHFR, but with lower affinity to eukaryotic forms of DHFR increasing the sensitivity of the three-hybrid system by reducing binding to endogenous DHFR.

### II. Linkers of Chemical Inducers of Dimerization

While the handles of a CID can be simply linked by a covalent bond between the two of them, more elaborate linkages or “linkers” may also be used depending on the screen to be performed. The linker should not significantly interfere with the reporting system, the affinities of the fused proteins for the DNA binding sites, or the affinities of the fused proteins for the handles on the CID. The linkage is formed by any of the methods known in the art (PCT International Publication No. WO 96/06097; Kathryn et al; House (1972); PCT International Publication No. WO 94/18317; PCT International Publication No. WO 95/02684; PCT International Publication No. WO 96/13613; PCT International Publication No. WO96/06097; and PCT International Publication No. WO 01/53355), these references being incorporated herein by reference. These linkers are all commercially available or can be prepared by available synthesis techniques. The linkers vary in hydrophobicity, length, and flexibility.

The linker is designed to respond to enzymatic activity. For example, a linker can contain a glycosidase bond, which is cleaved by a glycosidase enzyme and formed by a Glycosyltransferase enzyme; or an amide bond, which is cleaved by a protease and formed by peptidase or transpeptidase; or an aldol product bond, which is cleaved by a retro-aldolase and formed by aldolase; or an ester bond; or a phosphodiester bond. Such bonds can be used in bacterial based screens similarly to their use in yeast based screens, which are described in PCT International Publication No. WO 01/53355.

With a linker that contains an appropriate bond, the subject invention screens derivatives of large classes of enzymes.

A variety of enzymes and enzymes classes are listed on the World Wide Web beginning at prowl.rockefeller.edu/enzymes/enzymes.htm. All enzymes are given an Enzyme Commission (E.C.) number allowing it to be uniquely identified. E.C. numbers have four fields separated by periods, “a.b.c.d”. The left-hand-most field represents the broadest classification for the enzyme. The next field represents a finer division of that broad category. The third field adds more detailed information and the fourth field defines the specific enzyme. Thus, in the “a” field the classifications are oxidoreductases, transferases, hydrolases, lyases, isomerases, and ligases. Each of these “a” classifications are then further separated into corresponding “b”, each of which in turn is separated into corresponding “c” classifications, which are then further separated into corresponding “d” classes.

The subclasses of oxidoreductases are, for example: 1.1 CH—OH, 1.2 aldehyde or oxo, 1.3 CH—CH, 1.4 CH—NH₂, 1.5 CH—NH, 1.6 NADH OR NADPH, 1.7 other N-containing, 1.8 sulfur, 1.9 heme, 1.10 diphenols and related, 1.11 peroxidases, 1.12 hydrogen, 1.13 single donors+O₂, 1.14 paired donors+O₂, 1.15 superoxide radical, 1.16 oxidizing metal ions, 1.17 CH₂, 1.18 reduced ferredoxin, and 1.19 reduced flavodoxin.

The subclasses of transferases are, for example:

2.1 one carbon, 2.2 aldehydes or ketones, 2.3 acyl, 2.4 glycosyl, 2.5 alkyl or aryl, 2.6 N-containing, 2.7 P-containing, 2.8 S-containing, and 2.9 Se-containing.

The subclasses of hydrolases are, for example:

3.1 ester, 3.2 glycosidic, 3.3 ether, 3.4 peptide, 3.5 C—N (non-peptide), 3.6 acid anhydride, 3.7 C—C, 3.8 C-halide, 3.9 P—N, 3.10 S—N, 3.11 C—P, and 3.12 S—S.

The subclasses of lyases are, for example:

### 4.1 C—C, 4.2 C—O, 4.3 C—N, 4.4 C—S, 4.5 C-halide, and 4.6 P—O.

The subclasses of isomerase are, for example:

5.1 racemases and epimerases, 5.2 cis-trans isomerases, 5.3 intra-oxidoreductases, 5.4 intra-transferases (mutases), and 5.5 intramolecular lyases.

The subclasses of ligases are, for example:

### 6.1 C—O, 6.2 C—S, 6.3 C—N, 6.4 C—C, and 6.5 P-ester.

Each of the mentioned classes is further separated into sub, sub-classes, i.e. the “c” level, and then the “d” level. Moreover, new enzymes are discovered and are intended to be included within the scope of this invention, which is itself designed to evolve or discover such new enzymes.

The enzymes that act on such linkers are known enzymes or novel proteins which are screened for specific enzymatic activity. Novel enzymes can be evolved using combinatorial techniques. With a linker that contains an appropriate bond, the subject invention screens derivatives of large classes of enzymes.

One improvement to the linker is described in PCT

International Publication No. WO 02/070662, which improves the solubility and permeability of the linker. Since the handles on a CID often have low solubility, this addition of a more soluble linker increases the solubility of the entire CID. Thus, the CID is more readily available inside the cell which increases the sensitivity of the yeast three hybrid system.

### III. Synthesis of Chemical Inducers of Dimerization

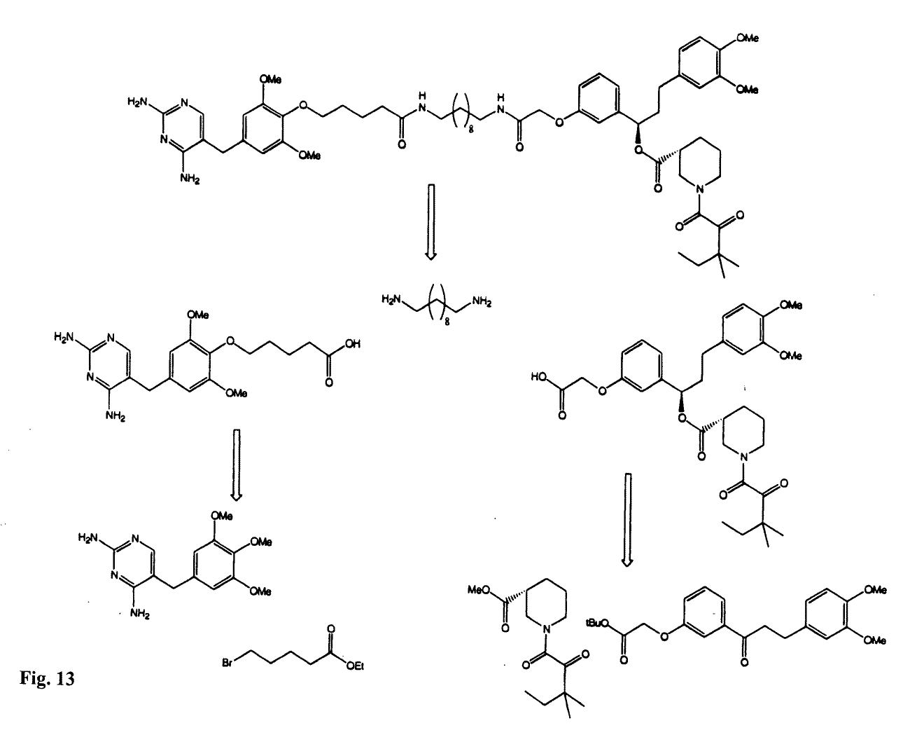

We have synthesized a trimethoprim-SLF CID molecule. The retrosynthesis is shown in FIG. 13. The carboxylic acid derivative of trimethoprim is first prepared. Trimethoprim was converted to a 4′-substituted phenol derivative by preferential cleavage of the 4′-methoxy group in hydrobromic acid. The phenol is then reacted with ethyl-5-bromovalerate in the presence of potassium tert-butoxide to yield a 4′-substituted ethyl ester. The ester is then deprotected in sodium hydroxide to yield the carboxylic acid derivative of trimethoprim. SLF acid was synthesized as described previously from L-pipecolinic acid in six steps. (Althoff et al. (2002); Amara et al. (1997); and Keenan et al. (1998)) The trimethoprim and SLF portions were then coupled to 1,10-diaminodecane in a three-component peptide coupling reaction.

This invention encompasses synthesis of trimethoprim-SLF CID molecules wherein the trimethoprim attaches to the linker at either one of the methoxy groups located at the meta position. Synthesis of these CID molecules incorporates all or portions of the synthesis described above.

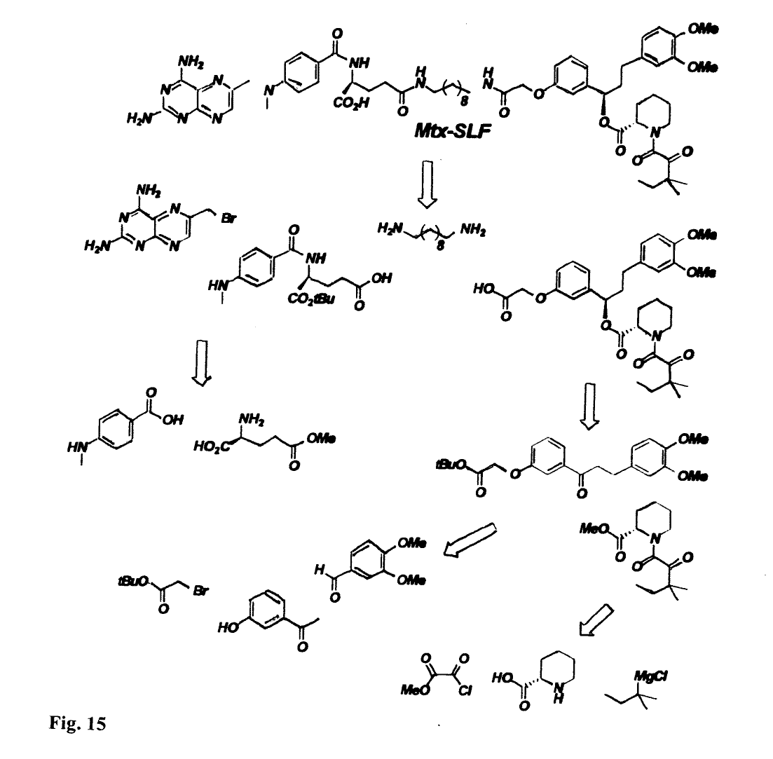

We have also synthesized a Mtx-SLF CID molecule. The retrosynthesis is shown in FIG. 15. The synthesis is designed to be modular so that we can easily bring in a variety of linkers in one of the final steps as the dibromo- or diiodo-derivatives. For synthetic ease, the glutamate residue has been replaced with homocysteine. This replacement should be neutral because there is both biochemical and structural evidence that the g-carboxylate of methotrexate can be modified without disrupting DHFR binding. The final compound has been synthesized in 12 steps in 1.3% overall yield. Also synthesized are analogous compounds where the a,a′-dibromo-m-xylene linker is replaced with 1,5-diiodopentane or 1,10-diiododecane. A similar route is used to prepare MTX-MTX molecules.

### IV. The Host Cell

The host cell for the three-hybrid system screen is any cell capable of expressing the protein or cDNA library of proteins to be screened. Some suitable host cells have been found to be yeast cells, such as Saccharomyces cerevisiae, and bacterial cells, such as E. coli.

### V. Design of Reporter Genes

A number of techniques exist for translating the dimerization of two proteins to an in vivo screen or selection. (Hu et al. (1990); Hu et al. (1995); Fields and Song (1989); Gyuris et al. (1993); Johnsson et al. (1994); Rossi et al. (1997); and Karimova et al. (1998))

A reporter gene assay measures the activity of a gene's promoter. It takes advantage of molecular biology techniques, which allow one to put heterologous genes under the control of a mammalian cell (Gorman et al. (1982); Alam and Cook (1990)). Activation of the promoter induces the reporter gene as well as or instead of the endogenous gene. By design the reporter gene codes for a protein that is easily detected and measured. Commonly it is an enzyme that converts a commercially available substrate into a product. This conversion is conveniently followed by either chromatography or direct optical measurement and allows for the quantification of the amount of enzyme produced.

Reporter genes are commercially available on a variety of plasmids for the study of gene regulation in a large variety of organisms (Alam and Cook, supra). Promoters of interest can be inserted into multiple cloning sites provided for this purpose in front of the reporter gene on the plasmid (Rosenthal (1987); Shiau and Smith (1988)). Standard techniques are used to introduce these genes into a cell type or whole organism (e.g., as described in Sambrook, J., Fritsch, E. F. and Maniatis, T. Expression of cloned genes in cultured mammalian cells. In: Molecular Cloning, edited by Nolan, C. New York: Cold Spring Harbor Laboratory Press, 1989). Resistance markers provided on the plasmid can then be used to select for successfully transfected cells.

Ease of use and the large signal amplification make this technique increasingly popular in the study of gene regulation. Every step in the cascade DNA->RNA->Enzyme->Product->Signal amplifies the next one in the sequence. The further down in the cascade one measures, the more signal one obtains.

In an ideal reporter gene assay, the reporter gene under the control of the promoter of interest is transfected into cells, either transiently or stably. Receptor activation leads to a change in enzyme levels via transcriptional and translational events. The amount of enzyme present is measured via its enzymatic action on a substrate.

It has also been established that enzyme activity can be monitored, in vivo, and new enzymes can be screened for, in vivo, by splitting peptides. (Baum et al. (1990); Smith et al. (1991); Kamada et al. (1998); and Hawkins et al. (1999)).

A “split ubiquitin” method, described in U.S. Pat. No. 5,585,245 and U.S. Pat. No. 5,503,977, detects protein-protein interactions by using an ubiquitin specific protease which cleaves a reporter polypeptide from a fusion protein. This cleavage of the reporter polypeptide is detected by several techniques.

### VI. Design of the Protein Chimeras

For a discussion of several types of protein chimeras see PCT International Publication No. WO 01/53355.

Protein chimeras can be constructed from the Brent two-hybrid system which uses LexA as the DNA-binding domain and B42 as the transcription activation domain. The full length LexA protein which includes both the N-terminal DNA-binding domain and the C-terminal dimerization domain is used. The B42 domain is a monomer. The reporter gene for this system is lacZ under control of 4 tandem LexA operators.

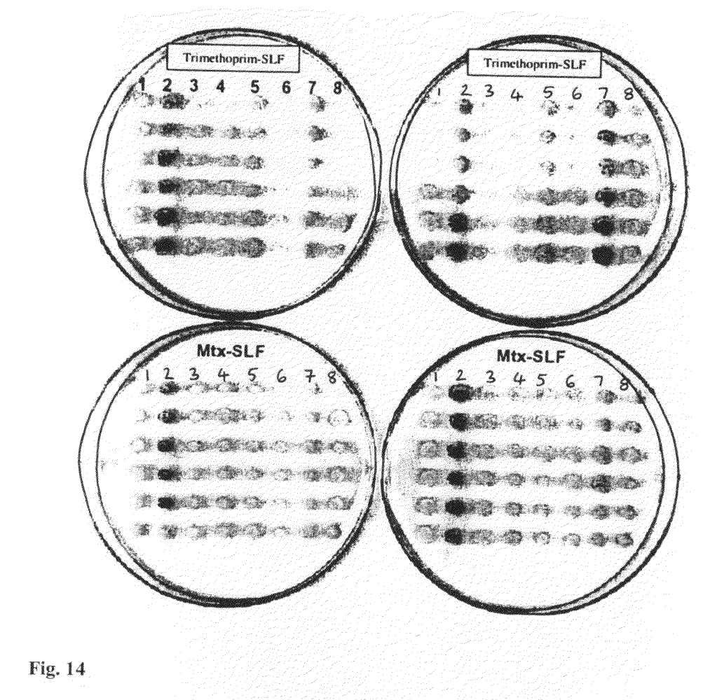

We chose to make a LexA-eDHFR and B42 fused to a series of FKBP12 mutants (see FIG. 14).

Construction of the LexA- and B42-receptor fusions is facilitated by the availability of commercial vectors for the Brent two-hybrid system. These vectors are shuttle vectors that can be manipulated both in bacteria and yeast. The LexA chimera is under control of the strong, constitutive alcohol dehydrogenase promoter. The B42 chimera is under control of the strong, regulatable galactose promoter. Both the FKBP12 mutants and the eDHFR gene can be introduced into the multiple cloning sites of the commercial LexA and B42 expression vectors using standard molecular biology techniques. The available restriction sites result in a three amino acid spacer between the two proteins in both the FKBP12 and the DHFR constructs. The plasmids encoding the LexA- and B42-fusion proteins can be introduced in all necessary combinations into S. cerevisiae strains containing a plasmid encoding the reporter gene.

While E. coli DHFR is one of the most well characterized DHFRs, other forms of DHFR may be used in this three-hybrid system.

In other embodiments of the invention, the DHFR used as one of the fused protein chimeras is of human or non-human origin. Non-limiting examples of DHFR proteins used according to the invention include proteins as described in Chang et al. (1978) (Mus musculus DHFR); Morandi et al. (Homo sapiens DHFR); Hao et al. (1994) (Drosophila DHFR); Fling and Richards (1983) (E. coli DHFR).

An embodiment of the present invention provides for the use of M. tuberculosis DHFR (or an active portion thereof) as fused protein chimera with a modified TMP as part of the CID that binds to the M. tuberculosis DHFR active site, but not to the human DHFR active site. (Li et al., (2000))

In another embodiment, Mycobacterium avium DHFR (or a portion thereof) is used as the fused protein chimera with a modified TMP as part of the CID with the TMP modified to selectively bind to M. avium DHFR (Rosowsky et al. (2002); and Zwyno-vanGinkel (1997)).

Other embodiments include using the DHFR of malarial parasites as the protein chimera, e.g., the DHFR of Plasmodium falciparum, (Bzik et al. (1987)), the DHFR of Toxoplasma gondii (Roos (1993)), the DHFR of Pneumocystis carinii (Edman et al. (1989)); and the DHFR of Tsypanosoma cruzi (Reche et al. (1994)).

In additional embodiments of the invention, the DHFR protein chimera is structurally modified to alter its ligand binding characteristics. For example, DHFR is altered to increase the size of its TMP binding site, as has been previously reported for methotrexate. (Clackson et al. (1998), Curr. Opin. Struct. Biol. 8:451-458; and Clackson et al. (1998), Proc. Natl. Acad. Sci. U.S.A. 95:10437-10442). If the TMP handle on the CID is also altered to bind to the larger DHFR binding site on the protein chimera, the altered TMP handle should bind weakly to wild-type DHFR and strongly to the altered DHFR.

Protein chimeras are varied in various ways including: inverting the orientation of the activation domain and the receptor; introducing tandem repeats of the receptor; introducing (GlyGlySer)ₙ linkers between the protein domains; and varying the DNA-binding domain and the transcription activation domain.

An approach to introducing tandem repeats of the receptor and (GlyGlySer)ₙ linkers that allows us to make multiple protein chimera constructs simultaneously is provided. The approach to making tandem repeats of the receptor is to use restriction enzymes with compatible cohesive ends. The same PCR product is then used to introduce each receptor unit. By including a BamHI restriction site immediately 5′ to the gene encoding the receptor, a series of (GlyGlySer)ₙ linkers is introduced essentially as described. This approach relies on the fact that the BamHI site, GGA-TCC, encodes Gly-Ser. This combined approach allows for the construction of multiple protein chimeras simultaneously. Since a lacZ screen is used, all of these constructs are assayed simultaneously.

### Uses of the Yeast Three-Hybrid System

The yeast three-hybrid system is broadly defined as a method of identifying ligand-receptor interactions. However, the yeast three-hybrid system can be modified and directed to accomplish more specific goals.

The yeast three-hybrid system is used to screen proteins for the ability to catalyze bond formation, comprising the steps of a) providing a cell that expresses a pair of fusion proteins which upon dimerization activate a cellular readout; b) providing a first compound and a second compound, each being capable of binding to one of the pair of fusion proteins, said first and second compound comprising a portion through which the first and second compounds are coupled by the action of the bond forming protein to be screened; and c) screening for the cellular readout, wherein a change in the cellular readout indicates catalysis of bond formation by the protein to be screened.

The yeast three-hybrid assay is also used to screen a compound for the ability to inhibit an enzyme comprising screening for activity of the enzyme by the method disclosed herein, and obtaining cells which express an active enzyme, and contacting the cells with the drug to be screened, wherein a change in the transcription of the reporter gene within the cell after contact with the drug indicates inhibition of the enzyme by the drug.

The yeast three-hybrid assay is also used to screen for a drug that inhibits an enzyme selected by this method.

The yeast three-hybrid assay is further used in a method of evolving a protein with a new catalytic activity comprising screening proteins derived from a library of proteins which are mutants of a known protein, using either of the screening methods provided by this invention.

Thus, this invention also provides a protein with new catalytic activity evolved by this method.

This invention also provides a method of evolving an enzyme with a new substrate specificity comprising screening enzymes derived from a library of enzymes which are mutants of an enzyme with known substrate specificity, using either of the screening methods provided by this invention.

Thus, this invention provides an engineered enzyme having new substrate specificity evolved by this method.

This invention also provides a method for evolving an enzyme that functions with a cofactor which is different from the cofactor the natural coenzyme uses, comprising evolving mutants of the natural coenzyme; and screening the mutants of the natural coenzyme in the presence of a cofactor different from the cofactor of the natural enzyme, using either of the screening methods provided by this invention.

### EXPERIMENTAL DETAILS

### Example 1

### Preparation of Mtx-SLF CID (the Control CID)

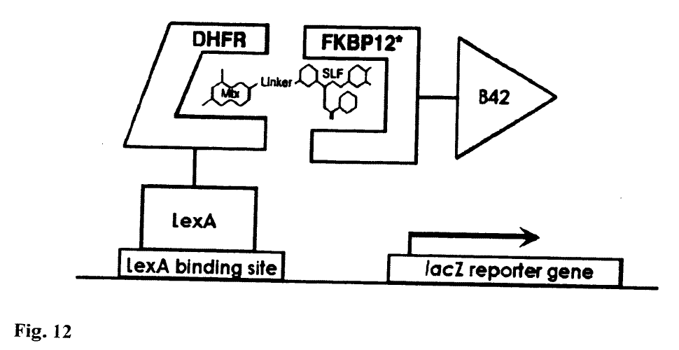

For the first of two bridging small molecules, a heterodimer of methotrexate (Mtx) and a synthetic analog of FK506 (SLF) was prepared. This heterodimer is referred to in this description Mtx-SLF. Mtx-SLF was used to dimerize the FKBP12 mutant protein chimeras fused to B42 with transcription activation domain and an E. coli dihydrofolate reductase (eDHFR) protein chimera fused to LexA DNA binding domain as shown in FIG. 12. Methotrexate (Mtx) inhibits dihydrofolate reductase (DHFR) with a low picomolar K_{I}, and the interaction between the two has been extensively studied (Bolin et al. (1982); and Benkovic et al. (1988)). For the other half of the bridging small molecule, we used SLF, available from Ariad Pharmaceuticals (Cambridge, Mass.) as an FK506 analog. SLF has nanomolar affinity for FKBP12, and the interaction between the two has been fully characterized (Keenan et al. (1998); and Holt et al. (1993)). In addition, SLF homodimers have been used previously in several mammalian three-hybrid systems (Clackson, (1997)).

The retrosynthetic analysis of Mtx-SLF is based on previous syntheses of Mtx and SLF derivatives and was designed to allow Mtx, SLF, or the linker between them to be varied readily. The Mtx portion of the molecule begins as the 7-methyl ester of L-glutamic acid and is based on previous syntheses of Mtx (Lin et al. (2000); Abida et al. (2002); and Keenan et al. (1998)). 7-Methyl L-glutamic acid is inexpensive, and the α-carboxylate can be selectively protected as the tert-butyl ester by transiently protonating the α-amino group (Liu et al. (1986)). The diprotected amino acid is then coupled to 4-(methylamino)benzoic acid using standard peptide coupling reagents. Finally, the γ-methyl ester is saponified to yield the free acid for further reactions. SLF acid was synthesized as described previously from L-pipecolinic acid in 59% yield for 6 steps (Amara et al. (1997); and Keenan et al. (1998)). The Mtx and SLF portions were then coupled to 1,10-diaminodecane in a three-component peptide coupling reaction. 2,4-Diamino-6-bromomethyl-pteridine is added after this coupling reaction in order to simplify purification of the synthetic intermediates. Finally, acid cleavage of the tert-butyl ester yielded Mtx-SLF. Thus, the Mtx-SLF heterodimer was prepared from two components in 5% overall yield for the 6 steps from the γ-methyl ester of L-glutamic acid or 6% overall yield in 9 steps from the L-pipecolinic acid precursor of SLF.

The synthetic chemistry performed for the preparation of the Mtx-SLF is described below.

### Synthetic Chemistry

Reagents were obtained from commercial suppliers and were used without further purification. All reagents for chemical synthesis were purchased from Aldrich. Anhydrous N,N-dimethylformamide and anhydrous methylene chloride were from Sure Seal™ bottles purchased from Aldrich. Methotrexate was a generous gift from the National Cancer Institute. Nuclear magnetic resonance (NMR) spectra were recorded on a Bruker 500 (500 MHz), a Bruker 400 (400 MHz) or a Bruker 300 (300 MHz) Fourier Transform (FT) NMR spectrometer at the Columbia University Chemistry Department NMR Facility. Spectra were determined in methanol-d₄ at 300 K with the proton or carbon (3.30 δ 49.0 δ) as the reference or in chloroform-d at 300K with proton (7.26 δ) as the reference. ¹H NMR spectra are tabulated in the following order: chemical shift calculated with reference to solvent standards based on tetramethylsilane, multiplicity (s, singlet; d, doublet; t, triplet; m, multiplet; br, broad), coupling constant(s) in Hertz, and number of protons. ¹³C NMR spectra were determined on the Bruker 300 MHz instrument and are proton decoupled. Mass spectra (MS) were recorded at the Columbia University Department of Chemistry mass spectral laboratory. Fast Atom Bombardment (FAB) high resolution mass spectra (HRMS) were recorded on a JMS-HX110A mass spectrometer. Low resolution electron spray ionization mass spectra (LRMS) were recorded on a JMS-LC Mate mass spectrometer. Analytical thin layer chromatography (TLC) was performed on silica gel (Whatman LHPKF Silica Gel 60 Å) and visualized by UV light (254 nm) or stained by ninhydrin. All column chromatography was flash chromatography carried out on silica gel (EM Science Silica Gel 60), and all eluants used are reported in volume:volume ratios. All moisture-sensitive reactions were performed under a positive pressure of nitrogen in flame- or oven-dried glassware. Organic extracts were dried over anhydrous sodium sulfate. Organic solvents were removed in vacuo with a rotary evaporator equipped with a vacuum pump (ca. 1 torr). Products were then left under vacuum (ca. 0.1 torr) overnight before analysis was performed.

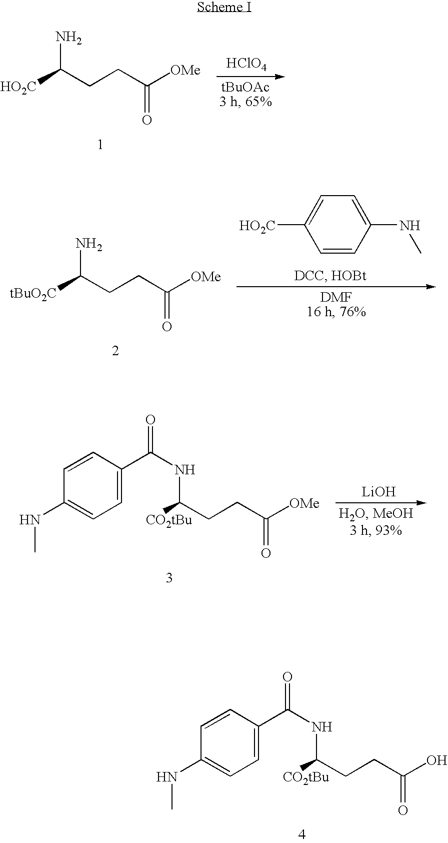

Synthesis of 2. (Liu et al. (1986)) The γ-methyl ester of L-glutamic acid (1) (5.02 g, 31.0 mmol) was added to a solution of 70% aqueous (aq.) perchloric acid (3.0 mL) in tert-butyl acetate (200 mL). The resulting solution was stirred at room temperature (rt) for 3 h during which time the acid dissolves completely. The reaction was then judged complete by thin layer chromatography (TLC) using 10:1 methylene chloride (CH₂Cl₂):methanol (MeOH). The reaction mixture was extracted with 0.5 N aq. HCl (4×, 400 mL). The pH of the combined aqueous layers was adjusted to 8 using saturated aq. sodium carbonate. The aqueous solution was then extracted with ethyl acetate (EtOAc) (4×, 500 mL). The organic extracts were combined, washed with brine (2×, 300 mL), and dried over anhydrous sodium sulfate. Removing the solvent in vacuo gave 2 as a clear oil in 65% yield: R_{f}=0.45 in 10:1 CH₂Cl₂:MeOH; ¹H NMR (400 MHz, CD₃OD) δ 3.66 (s, 3), 3.33 (t, J=6.5 Hz, 1), 2.41 (m, 2), 1.95 (m, 1), 1.86 (m, 1); LRMS, m/z 218.2 (MH⁺), 219.2 (MH2⁺).

Synthesis of 3. (Lin et al. (2000); Hart et al. (1996)) Compound 2 (2.19 g, 10.0 mmol), 1,3-dicyclohexylcarbodiimide (DCC, 3.09 g, 15.0 mmol), 1-hydroxylbenzotriazole hydrate (HOBt, 2.43 g, 18.0 mmol), and N-methyl-para-benzoic acid (1.59 g, 10.5 mmol) were dissolved in anhydrous dimethyl formamide (DMF, 22 mL) under a nitrogen atmosphere. Diisopropylethylamine (DIEA, 0.1 mL, 0.5 mmol) was added to the solution, and the reaction mixture was stirred overnight (ON) at rt. After 16 hr, a 1:2:1 water:saturated aq. sodium bicarbonate:brine solution (500 mL) was added to the reaction giving a yellow suspension. This solution was then extracted with EtOAc (4×, 300 mL). The fractions were combined, washed with brine (2×, 200 mL) and dried over anhydrous sodium sulfate. The organic solvent was then removed in vacuo. The product is purified by silica gel column chromatography (2:1 to 1:1 hexanes:EtOAc) in 76% yield: R_{f}=0.25 in 1:1 hexanes:EtOAc; ¹H NMR (500 MHz, CD₃OD) δ 7.66 (d, J=7.0 Hz, 2), 6.58 (d, J=7.0 Hz, 2), 4.59 (dd, J=9.5, 5.0 Hz, 1), 3.65 (s, 3), 2.79 (s, 3), 2.47 (t, J=7.5 Hz, 2), 2.23 (m, 1), 2.05 (m, 1), 1.46 (s, 9); ¹³C NMR (300 MHz, CD₃OD) δ 175.1, 173.0, 170.6, 154.6, 130.2, 121.5, 111.9, 82.9, 54.3, 52.3, 31.4, 30.0, 28.3, 27.6; LRMS, m/z 351.2 (MH⁺); HRMS, m/z 351.1930 (MH⁺), calculated 351.1920.

Synthesis of 4. Compound 3 (500 mg, 1.43 mmol) was dissolved in methanol (20 mL). Lithium hydroxide monohydrate (120 mg, 2.86 mmol) was dissolved in water. Both solutions were chilled in a 0° C. ice bath. The aqueous solution was added to the methanol solution all at once. The resulting solution was stirred at 0° C. for 10 minutes and then allowed to warm to rt and stirred for an additional 80 minutes. Solvent is removed in vacuo until only a yellow gel remained with a volume about 1 mL. Water (20 mL) was added to the remaining reaction mixture. The solution was acidified to pH=2 with 1 N aq. HCl (9 mL) and was immediately extracted with EtOAc (5×, 25 mL). The organic extracts were combined, washed with brine (2×, 20 mL), and dried over anhydrous sodium sulfate. The solvent was removed in vacuo to yield product 4 in 93% yield: R_{f}=0.05 in 1:1 EtOAc:hexanes and 0.45 in 5:1 CH₂Cl₂:MeOH; ¹H NMR (400 MHz, CD₃OD) δ 7.64 (d, J=7.0 Hz, 2), 6.56 (d, J=7.0 Hz, 2), 4.46 (dd, J=9.0, 5.0 Hz, 1), 2.80 (s, 3), 2.44 (t, J=7.5 Hz, 2), 2.23 (m, 1), 2.05 (m, 1), 1.47 (s, 9); ¹³C NMR (300 MHz, CD₃OD) δ 178.4, 174.8, 172.3, 156.2, 130.7, 123.2, 113.6, 84.6, 57.0, 33.2, 31.8, 30.5, 29.1; LRMS, m/z 337.3 (MH⁺); HRMS, m/z 337.1751 (MH⁺), calculated 337.1763.

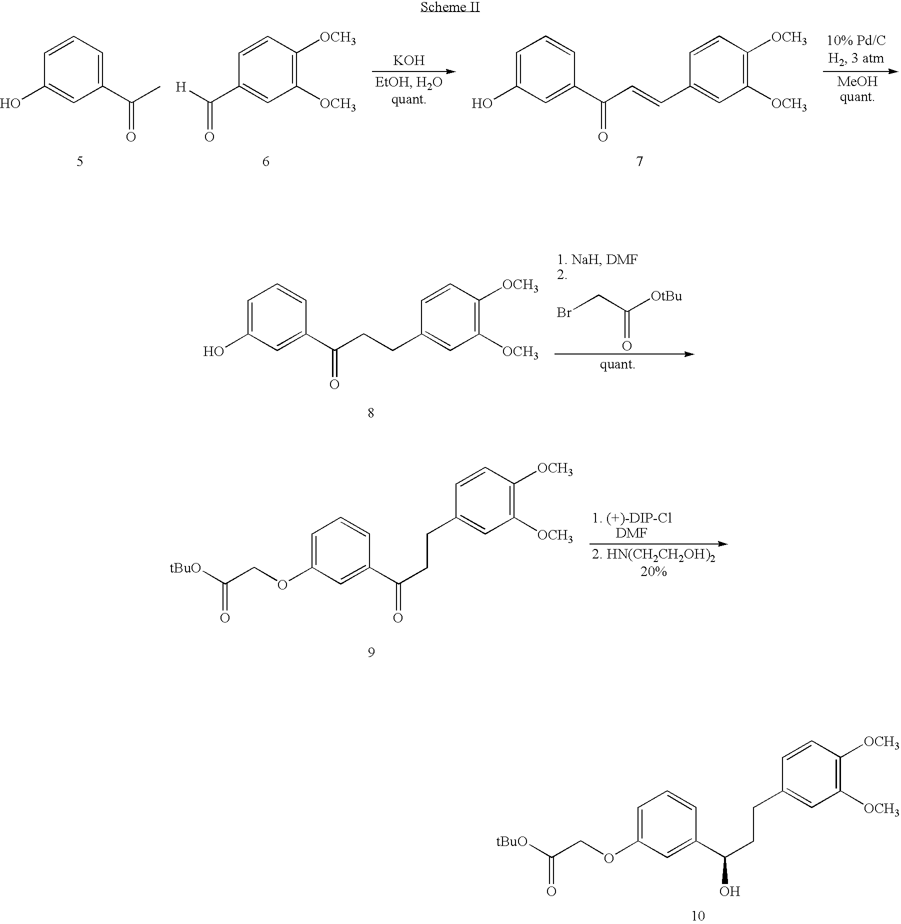

Synthesis of 7. (Keenan et al. (1998)) Synthesized as reported in quantitative yield to give 7 as a yellow solid: R_{f}=0.40 in 1:1 EtOAc:hexanes; ¹H NMR (300 MHz, CDCl₃) δ 9.86 (s, 1), 7.61-7.55 (m, 2), 7.40-7.34 (m, 2), 7.32 (d, J=4.5 Hz, 1), 7.25-7.10 (m, 2), 6.91-6.85 (m, 2), 3.95 (s, 3), 3.93 (s, 3); LRMS, m/z 284.9 (MH⁺).

Synthesis of 8. (Keenan et al. (1998)) Synthesized as reported in near quantitative yield based on mass as a white crystalline solid (NMR revealed a small amount of non-hydrogenated starting material remains and is carried through to the next step. NMR integration is used to determine relative quantities of the two materials.): R_{f}=0.50 in 1:1 EtOAc:hexanes; ¹H NMR (300 MHz, CDCl₃); δ 7.55-7.48 (m, 2), 7.35 (t, J=8.0 Hz, 1), 7.25 (t, J=8.5 Hz, 1), 7.10 (br d, J=8.0 Hz, 1), 6.91-6.73 (m, 3), 3.90 (s, 3), 3.88 (s, 3), 3.24 (t, J=7.5 Hz, 2), 2.99 (t, J=8.0 Hz, 2); LRMS, m/z 287.0 (MH⁺).

Synthesis of 9. (Keenan et al. (1998)) Synthesized as reported to give 9 as a clear oil in quantitative yield by mass: R_{f}=0.60 in 1:1 EtOAc:hexanes; ¹H NMR (300 MHz, CDCl₃) δ 7.59 (br d, 8.0 Hz, 1), 7.49 (m, 1), 7.37 (t, J=8.0 Hz, 1), 7.14 (dd, J=8.0, 2.5 Hz, 1), 6.85-6.72 (m, 3), 4.60 (s, 2), 3.89 (s, 3), 3.86 (s, 3), 3.27 (t, J=7.1 Hz, 2), 3.04 (t, J=7.5 Hz, 2), 1.50 (s, 9).

Synthesis of 10. (Keenan et al. (1998)) Synthesized as reported to yield product 10 in 20% yield fully purified. However, a 75% yield of unreacted starting material was also recovered. R_{f}=0.40 in 5:1 CH₂Cl₂:MeOH and 0.40 in 1:1 EtOAc:hexanes; ¹H NMR (500 MHz, CD₃OD) δ 7.28 (t, J=8.0 Hz, 1), 6.93 (d, J=7.5 Hz, 1), 6.89 (s, 1), 6.82 (d, J=8.0 Hz, 1), 6.80-6.74 (m, 2), 6.70 (d, J=8.0 Hz, 1), 4.60-4.53 (m, 3), 3.80 (s, 3), 3.78 (s, 3), 2.65-2.51 (m, 2), 2.04-1.89 (m, 2), 1.47 (s, 9); LRMS, m/z 403.3 (MH⁺).

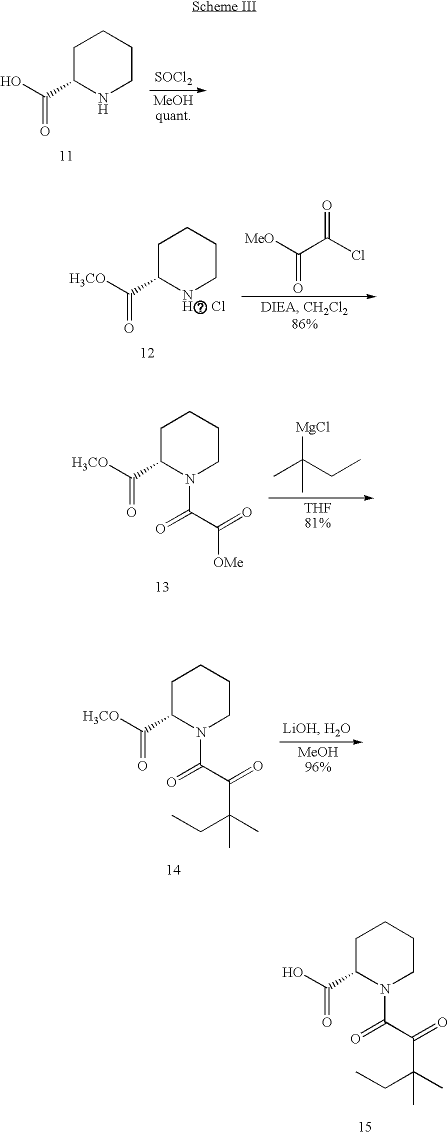

Synthesis of 12. (Holt et al. (1993)) Synthesized as reported to give 12 as a white crystalline solid in quantitative yield based on NMR integration: ¹H NMR (400 MHz, CD₃OD) δ 4.03 (br d, J=9.5 Hz, 1), 3.83 (s, 3), 3.41 (br d, J=12.0 Hz, 1), 3.04 (br t, J=11.0 Hz, 1), 2.27 (br d, J=11.5 Hz, 1), 2.00-1.63 (m, 5).

Synthesis of 13. (Holt et al. (1993)) Synthesized as reported to yield 13 as a clear oil in 86% yield: R_{f}=0.70 in 1:1 EtOAc:hexanes and 0.25 in 1:4 EtOAc:hexanes; ¹H NMR (500 MHz, CD₃OD) δ 5.15 (br d, J=5.0 Hz, 0.7), 4.62 (br d, J=4.0 Hz, 0.3), 4.34 (br d, J=12.0 Hz, 0.3), 3.89 (s, 2.1), 3.84 (s, 0.9), 3.77 (s, 3), 3.57 (br d, J=14.0 Hz, 0.7), 3.35 (m, 0.7), 2.91 (br t, J=13.0 Hz, 0.3), 2.35-2.24 (m, 1), 1.83-1.65 (m, 3) 1.55-1.35 (m, 2) This product exists as a 2.5:1 mixture of the trans and cis conformations. Further analysis by COSY allows us to make the following assignments of the two peaks for each proton in the structure: 5.15 and 4.62, 4.34 and 3.57, 3.89 and 3.84, 3.35 and 2.91; LRMS, m/z 230.1 (MH₁⁺).

Synthesis of 14. (Holt et al. (1993)) Synthesized as reported to give 14 as a clear oil in 81% yield: R_{f}=0.85 in 1:1 EtOAc:hexanes and 0.50 in 1:4 EtOAc:hexanes; ¹H NMR (500 MHz, CD₃OD) δ 5.16 (br d, J=5.5 Hz, 0.8), 4.38 (br d, J=11.5 Hz, 0.2), 4.24 (br d, J=5.0 Hz, 0.2), 3.75 (s, 3), 3.39 (br d, j=13.0 Hz, 0.8), 3.23 (td, J=13.0, 2.8 Hz, 0.8), 2.91 (br t, J=13.0 Hz, 0.2), 2.30 (br d, J=14.0 Hz, 0.8), 2.22 (br d, J=13.5, 0.2), 1.79-1.60 (m, 5) 1.55-1.35 (m, 2), 1.23-1.13 (m, 6), 0.86 (t, J=7.5 Hz, 3) This product exists as a 4:1 mixture of the trans and cis conformations. Further analysis by COSY allows us to make the following assignments of the two peaks for each proton in the structure: 5.16 and 4.24, 4.38 and 3.39, 3.23 and 2.91, 2.30 and 2.22; LRMS, m/z 270.2 (MH⁺).

Synthesis of 15. (Holt et al. (1993)) Synthesized as reported to give the product 15 as a white crystalline material in 96% yield: R_{f}=0.05 in 1:1 EtOAc:hexanes; ¹H NMR (500 MHz, CDCl₃) δ 5.29 (br d, J=5.5 Hz, 0.8), 4.50 (br d, J=14.0 Hz, 0.2), 4.26 (br d, J=4.5 Hz, 0.2), 3.43 (br d, J=13.0 Hz, 0.8), 3.24 (td, J=12.5, 3.5 Hz, 0.8), 2.94 (br td, J=13.0, 3 Hz, 0.2), 2.34 (br d, J=12.0 Hz, 0.8), 2.24 (br d, J=13.5, 0.2), 1.79-1.60 (m, 5) 1.55-1.35 (m, 2), 1.23-1.13 (m, 6), 0.86 (t, J=7.5 Hz, 3) This product exists as a 4:1 mixture of the trans and cis conformations. Further analysis by COSY allows us to make the following assignments of the two peaks for each proton in the structure: 5.29 and 4.24, 4.50 and 3.43, 3.24 and 2.94, 2.34 and 2.24. Compounds 16, 17, 18, and Mtx-SLF all have this same 4:1 conformation pattern and appear nearly the same spectroscopically as compound 15 for these peaks. For simplicity's sake the 0.8 integration is called 1 and the 0.2 peak is disregarded in the characterization for the rest of these compounds.

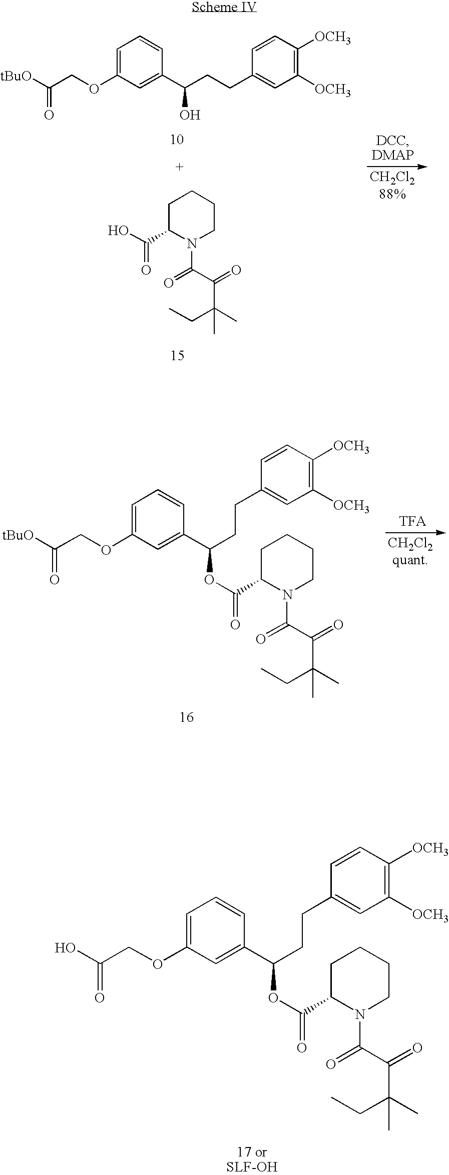

Synthesis of 16. (Keenan et al. (1998)) Synthesized as reported to give 16 as a colorless oil in 88% yield: R_{f}=0.15 in 4:1 hexanes:EtOAc and 0.65 in 1:1 EtOAc:hexanes; ¹H NMR (500 MHz, CD₃OD) δ 7.28 (t, J=8.0 Hz, 1), 6.96 (m, 1), 6.89 (s, 1), 6.85 (m, 2), 6.78 (s, 1), 6.71 (d, J=8.0 Hz, 1), 5.73 (m, 1), 5.21 (br d, J=5.5 Hz, 1), 4.58 (s, 2), (s, 3), 3.78 (s, 3), 3.38 (br d, J=14.5 Hz, 1), 3.17 (td, J=13.0, 3.0 Hz, 1), 2.65-2.52 (m, 2), 2.32 (br d, J=13.0 Hz, 1), 2.30-2.20 (m, 1), 2.05 (p, J=7.0 Hz, 1), 1.78-1.58 (m, 5), 1.46 (s, 9), 1.41-1.26 (m, 2), 1.23 (s, 3), 1.21 (s, 3), 0.86 (t, J=7.5 Hz, 3) (see note in Compound 15); LRMS, m/z 640.7 (MH⁺).

Synthesis of 17. (Keenan et al. (1998)) Synthesized as reported to give 17 in quantitative yield. TLC analysis showed only one product and the acid was used without further purification: R_{f}=0.05 in 1:1 EtOAc:hexanes and 0.35 in 10:1 CH₂Cl₂:MeOH.

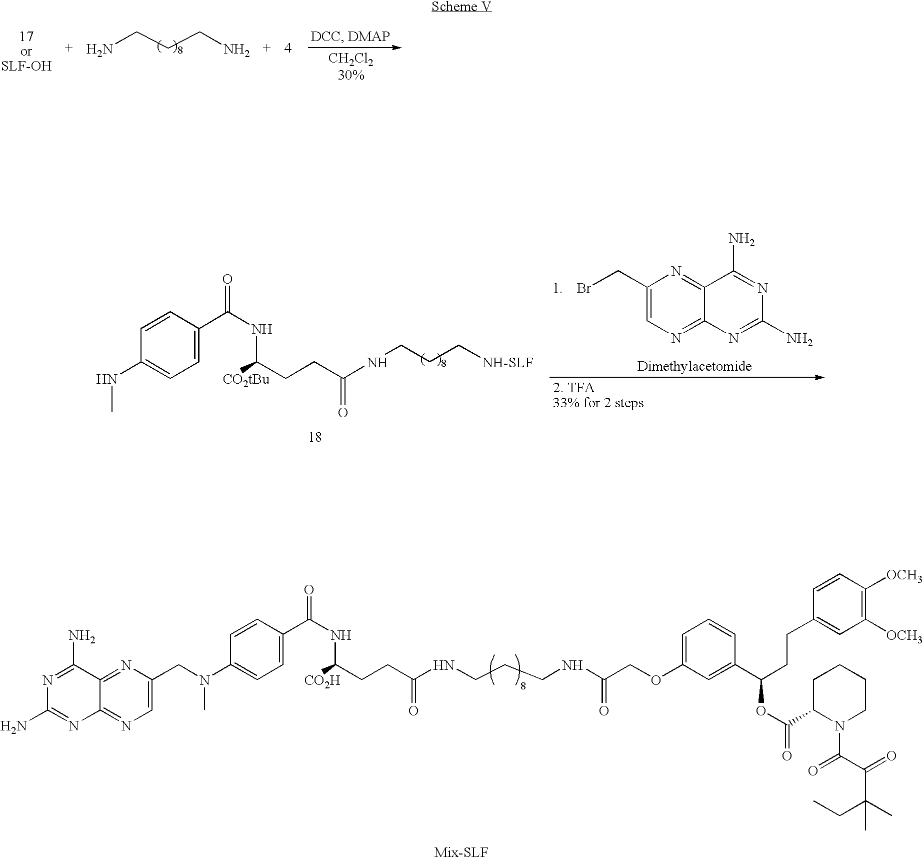

Synthesis of 18. Compound 4 (42.7 mg, 0.127 mmol), compound 17 (74.1 mg, 0.127 mmol), 1,10-diaminodecane (20.7 mg, 0.120 mmol), DCC (130 mg, 0.631 mmol), and DMAP (30.0 mg, 0.246 mmol) were dissolved in CH₂Cl₂ (3 mL) under a nitrogen atmosphere and stirred at rt overnight. After 16 hours, the solvent was removed in vacuo. The product was purified by silica gel column chromatography (1:4 EtOAc:hexanes to pure EtOAc) to give a white solid in 30% yield: R_{f}=0.65 in EtOAc and 0.60 in 10:1 CH₂Cl₂:MeOH; ¹H NMR (500 MHz, CD₃OD) δ 7.64 (d, J=7.0 Hz, 2), 7.28 (td, J=8.0, 3.0 Hz, 1), 6.98 (m, 2), 6.91 (d, J=7.5 Hz, 1), 6.85 (d, J=8.0 Hz, 1), 6.76 (s, 1), 6.69 (d, J=8.0 Hz, 1), 6.56 (d, J=7.0 Hz, 2), 5.74 (m, 1), 5.21 (br d, J=5.0 Hz, 1), 4.51 (s, 2), 4.46 (m, 1), 3.80 (s, 3), 3.78 (s, 3), 3.38 (br d, J=12.5 Hz, 1), 3.23 (t, J=7.0 Hz, 2) 3.17 (td, J=13.0, 3.0 Hz, 1), 3.11 (t, J=7.0 Hz, 2), 2.80 (s, 3); 2.65-2.52 (m, 2), 2.37-2.30 (m, 3), 2.30-2.15 (m, 2), 2.10-2.00 (m, 2), 1.78-1.58 (m, 4), 1.48 (s, 9), 1.54-1.38 (m, 5), 1.38-1.28 (m, 2), 1.28-1.16 (m, 18), 0.86 (t, J=7.5 Hz, 3) (see note in Compound 15).

Synthesis of Mtx-SLF. (Lin et al. (2000); Abida et al. (2002); Hart et al. (1996)) Compound 18 (40.0 mg, 38.0 mol) and the hydrobromide salt of 2,4-diamino-6-bromomethyl pteridine (18 mg, 45 mol) were dissolved in N,N′-dimethyl acetamide (2 mL). The reaction mixture was stirred in a 50° C. oil bath for 12 hours. The intermediate product (R_{f}=0.50 in 10:1 CH₂Cl₂:MeOH) was purified by silica gel column chromatography (30:1 to 10:1 CH₂Cl₂:MeOH). The crude product was dissolved in trifluoroacetic acid (3 mL) at 0° C. for 5 minutes and allowed to warm to rt and stirred at rt for 1 hour. Toluene (3×, 50 mL) was added to the reaction mixture, and all solvent was removed in vacuo. After removal of solvents, Mtx-SLF was purified by preparative thin layer silica gel chromatography (5:1 CH₂Cl₂:MeOH, 4×) to give a yellow solid in 33% yield (for two steps): R_{f}=0.15 in 3:1 CH₂Cl₂:MeOH; ¹H NMR (500 MHz, CD₃OD) δ 8.55 (s, 1), 7.73 (d, J=8.0 Hz, 2), 7.28 (t, J=7.5 Hz, 1), 6.98 (m, 2), 6.91 (br d, J=7.5 Hz, 1), 6.85-6.79 (m, 3), 6.76 (s, 1), 6.69 (d, J=6.5 Hz, 1), 5.73 (m, 1), 5.21 (br s, 1), 4.75 (s, 2), 4.50 (s, 2), 4.46 (m, 1), 3.80 (s, 3), 3.78 (s, 3), 3.38 (br d, J=13.0 Hz, 1), 3.25-3.10 (m, 6), 3.05 (t, J=7.0 Hz, 2), 2.65-2.52 (m, 2), 2.37-2.15 (m, 5), 2.10-2.00 (m, 2), 1.78-1.58 (m, 4), 1.50-1.42 (m, 3), 1.38-1.32 (m, 3), 1.35-1.25 (m, 4), 1.25-1.16 (m, 15), 0.86 (t, J=7.5 Hz, 3) (see note in Compound 15).

### Preparation of Trimethoprim-SLF

For the second CID, a heterodimer of trimethoprim and a synthetic analog of FK506 (SLF) was prepared. This heterodimer is referred to in this description as trimethoprim-SLF. Trimethoprim-SLF was used to dimerize the FKBP12 mutant protein chimeras fused to B42 with transcription activation domain and an E. coli dihydrofolate reductase (eDHFR) protein chimera fused to LexA DNA binding domain. Trimethoprim selectively inhibits eDHFR. Trimethoprim has a high affinity for eDHFR, but a low affinity for the mammalian form of DHFR. (Baccanari et al.)

In the retrosynthetic analysis of trimethoprim-SLF, the carboxylic acid derivative of trimethoprim was first prepared. Trimethoprim was converted to a 4′-substituted phenol derivative by preferential cleavage of the 4′-methoxy group in hydrobromic acid. The phenol is then reacted with ethyl-5-bromovalerate in the presence of potassium tert-butoxide to yield a 4′-substituted ethyl ester. The ester is then deprotected in sodium hydroxide to yield the carboxylic acid derivative of trimethoprim. SLF acid was synthesized as described previously from L-pipecolinic acid in six steps. (Althoff et al. (2002), Amara et al. (1997), and Keenan et al. (1998)) The trimethoprim and SLF portions were then coupled to 1,10-diaminodecane in a three-component peptide coupling reaction.

CID concentration calibration. Both CIDs were dissolved in DMF to concentrations of 12 mM for the Mtx-SLF molecule. The concentrations of Mtx and Mtx-SLF were determined by Beer's law using an extinction coefficient of =6700 cm⁻¹M⁻¹ (calculated from a known solution of Mtx in DMF) for Mtx-SLF. Solutions of compound 16 (SLF-OtBu) were prepared on a sufficient scale to mass 16 accurately. All CIDs were stored under a nitrogen atmosphere at −80° C. and allowed to come to rt before use.

### Materials for Yeast Three-Hybrid System.

Standard protocols for molecular biology and yeast genetics were used. Restrictions enzymes and T4 DNA ligase were purchased from New England Biolabs (Beverly, Mass.). PFU Turbo polymerase and TG1 cells were purchased from Stratagene (La Jolla, Calif.). The dNTPs used for PCR reactions were purchased from Amersham Pharmacia Biotech. The Tuner BL21(DE3) cells, pET26b plasmid, and the BugBuster protein extraction reagent used in protein purification were purchased from Novagen (Madison, Wis.). The yeast protein extraction reagent, Y-Per, was purchased from Pierce (Rockford, Ill.). The 5-bromo-4-chloro-3-indoyl β-D-galactopyranoside (Xgal) used in the X-gal indicator plates was purchased from Diagnostic Chemicals (Oxford, Conn.). The phrog used to transfer 48 droplets of yeast media onto X-gal plates was purchased from Dan-Kar Corp. (Wilmington, Mass.). The Ni-NTA spin columns used to purify proteins as well as the miniprep spin kit and the gel extraction spin kit used to purify DNA were purchased from Qiagen (Valencia, Calif.). Centricon centrifugal filter units YM-3 for protein concentration and buffer change were purchased from Millipore (Billerica, Mass.). Anti-HA IgG antibodies for protein detection were purchased from Roche (Indianapolis, Ind.). Anti-FKBP12 IgG antibodies for protein detection was purchased from ABR Affinity Bio Reagents (Golden, Colo.). Anti-LexA IgG for protein detection was purchased from Invitrogen Corp. (Carlsbad, Calif.). The ECF Western blotting reagent pack, which included the anti-mouse and anti-rabbit IgG-linked alkaline phosphatase, was purchased from Amersham Pharmacia Biotech. Oligonucleotides were purchased from the Great American Gene Co. (Ramona, Calif.). Isopropyl β-D-thiogalactoside (IPTG) was purchased from American Bio-Organics. N,N,N′,N′-Tetramethylethylenediamine (TEMED) and acrylamide:bisacrylamide (37:5:1) for making 15% acrylamide/bisacrylamide gels were purchased from Fisher (Pittsburgh, Pa.). Methotrexate was a gift from the National Cancer Institute (NCI). All other chemicals were purchased from Aldrich or Molecular Probes. All solutions were made from distilled water prepared from a Milli Q water purification system. For PCR, a MJ Research PTC-200 Pellier thermal cycler was used.

### Plasmid Construction.

For expression of the B42-FKBP12 fusion in the yeast three-hybrid system, we created plasmid pMW102-FKBP12. A 345 bp EcoRI to XhoI fragment encoding the FKBP12 gene was created by using the primers VWC22, 5′-GCA TAC GTC GAA TTC ATG GGA GTG CAG GTGG (EcoRI), and VWC23, 5′-GCA TTG CTG CTC GAG TCA TTC CAG TTT TAG AAG C (XhoI). This fragment was inserted between the EcoRI and XhoI sites in the pMW102 vector to generate the pMW102-FKBP12 plasmid, encoding FKBP12 fused to the C-terminus of the HA epitope and B42. The FKBP12 mutants used in both the overexpression vector and the yeast three-hybrid vector were made using Stratagene's QuikChange site-directed mutagenesis kit. The following primers were used to make the FKBP12 variants. For D37V the primers VWC1022, 5′-GAA GAT GGA AAG AAA TTT GTT TCC TCC CGG GAC AGA AAC, and VWC1023, 5′-GTT TCT GTC CCG GGA GGA AAC AAA TTT CTT TCC ATC TTC, generated plasmids pBC1052 from pMW102-FKBP12 and pKS1274 from pSG1205. For W59L the primers VWC1364, 5′-GTG ATC CGA GGC TTG GAA GAA GGG GTT GCC, and VWC1365, 5′-GGC AAC CCC TTC TTC CAA GCC TCG GAT CAC, generated plasmids pKS1269 from pMW102-FKBP12 and pKS1275 from pSG1205. For F36Y the primers VWC1366, 5′-GAA GAT GGA AAG AAA TAT GAT TCC TCC CGG GAC AG, and VWC1367, 5′-CT GTC CCG GGA GGA ATC ATA TTT CTT TCC ATC TTC, generated plasmids pKS1270 from pMW102-FKBP12 and pKS1276 from pSG1205. For R42Q the primers VWC1015, 5′-AAA TTT GAT TCC TCC CGG GAC CAA AAC AAG CCC TTT AAG TTT ATG, and VWC1016, 5′-CAT AAA CTT AAA GGG CTT GTT TTG GTC CCG GGA GGA ATC AAA TTT, generated plasmids pBC1053 from pMW102-FKBP12 and pKS1277 from pSG1205. For H87L the primers VWC1017, 5′-GCC TAT GGT GCC ACT GGG TTG CCA GGC ATC ATC CCA CCA, and VWC1018, 5′-TGG TGG GAT GAT GCC TGG CAA CCC AGT GGC ACC ATA GGC, generated the plasmids pBC1054 from pMW102-FKBP12 and pKS1278 from pSG1205. And for Y26F the primers VWC1372, 5′-ACC TGC GTG GTG CAC TTC ACC GGG ATG CTT GAA, and VWC1373, 5′-TTC AAG CAT CCC GGT GAA GTG CAC CAC GCA GGT, generated plasmids pKS1273 from pMW102-FKBP12 and pKS1279 from pSG1205. The full coding region was sequenced by using the primers VWC422, 5′-CAA GAC CCG TTT AGA GGC, for the plasmids derived from pSG1205 and VWC1053, 5′-CAG CCT CTT GCT GAG TGG, for the plasmids derived from pMW102-FKBP12.

### Strain Construction.

The yeast strains used in this experiment were all derived from parent strain V704Y. The strain V704Y was prepared by integrating the gene encoding LexA-DHFR under the control of the GAL1 promoter at the chromosomal loci ade4 in the S. cerevisiae strain FY251 (MATa). This strain was transformed using lithium acetate with the plasmid pMW112, which encodes the lacZ gene under the control of eight tandem LexA operators, and the one of the following eight plasmids pMW102 (encoding B42, negative control), pMW102-FKBP12 (encoding B42-FKBP12), pBC1052 (encoding B42-FKBP12 with D37V mutation), pKS1269 (encoding B42-FKBP12 with W59L mutation), pKS1270 (encoding B42-FKBP12 with F36Y mutation), pBC1053 (encoding B42-FKBP12 with R42Q mutation), pBC1054 (encoding B42-FKBP12 with H87L mutation), or pKS1273 (encoding B42-FKBP12 with Y26F mutation) followed by selection on synthetic complete (SC) media containing 2% glucose and lacking the appropriate selective nutrients as described (Baker et al. 2002). Six transformants for each mutant were picked for use in the yeast three-hybrid system.

### Assays.

The yeast strains expressing the three-hybrid constructs with wild-type and mutant versions of FKBP12 were assayed for β-galactosidase activity both on plates and on liquid cultures. Each of the yeast strains was inoculated from frozen glycerol stocks onto SC media containing 2% glucose and lacking the appropriate nutrients. Incubation at 30° C., shaking at 250 rpm, was allowed to proceed until saturation (approximately 3 days). Plate assays were performed by transferring a droplet of the saturated yeast culture onto X-gal indicator plates, using a 48 prong phrogger. The X-gal indicator plates contained either 0, 1, or 10 μM Mtx-SLF or trimethoprim-SLF along with 0.5% glucose, 1.5% galactose, and 2% raffinose and lacked the appropriate selective nutrients in order to induce expression of the LexA-DHFR and B42-FKBP12 chimeras, which were under the control of the GALL promoter. Competition experiments were also performed using X-gal indicator plates containing 1 μM Mtx-SLF in the presence of 0, 10, or 100 μM SLF or 1 μM trimethoprim-SLF in the presence of 0, 10, or 100 μM SLF. The plates were allowed to grow at 30° C. for 3-5 days. For the liquid assays, 5 μL of the saturated yeast cultures was used to inoculate 95 μL of SC media containing 0.5% glucose, 1.5% galactose, and 2% raffinose and lacking the appropriate. Identical inoculations were done except that the media contained varying concentrations (0.01-30 μM) of the Mtx-SLF or trimethoprim-SLF. The cultures were allowed to grow for 3 days at 30° C., shaking at 250 rpm. The cells were harvested by centrifugation, and the pellets were subsequently resuspended in 100 μL of distilled water. Next, the cultures were transferred to a flat-bottomed 96-well plate, (Fisher, Pittsburgh, Pa.) for reading at A₆₀₀. The cultures were centrifuged, and the pellets were resuspended in 100 μL of the Y-Per protein extraction reagent. Lysis was allowed to proceed for 30 min. Then, 8.5 μL of a 10 mg/mL ONPG solution was added to the extracts and allowed to incubate for 30 min at 37° C., shaking at 300 rpm. The β-galactosidase activity reaction was stopped with 130 μL of 1 M sodium carbonate. The extracts were centrifuged, and the supernatant was transferred to a flat-bottomed 96-well plate, where the A₄₂₀ was measured. The following equation was used to calculate β-galactosidase units: β-galactosidase=1000[A₄₂₀/(A₆₀₀×time (in min)×volume assayed (in mL))].

### Results

Trimethoprim-SLF was used to activate transcription in the Y3H system described in FIG. 12. Trimethoprim-SLF was compared directly against Mtx-SLF for its ability to induce lacZ transcriptions in yeast strains expressing LexA-eDHFR and B42 fused to a series of FKBP12 mutants (see FIG. 14). The wild-type FKBP12-containing strains (column 2) all showed a clear blue coloration in the presence of both 1 and 10 μM Mtx-SLF and trimethoprim-SLF. After 3 days of growth, the trimethoprim-SLF showed stronger activation than the Mtx-SLF, as evidenced by activation of mutant number 5 (encoding B42-FKBP12 with F36Y mutation, and stronger activation of mutant number 7 (encoding B42-FKBP12 with H97L mutation) (FIG. 14). Strains possessing R42Q (column 6), W59L (column 4), D37V (column 3), and Y26F (column 8) all failed to show any blue coloration in the plate assays.

### Example 2

We hypothesized that the cross-reactivity of Mtx with endogenous DHFR in the yeast cells could impair transcription activation by Dex-Mtx in the yeast three-hybrid assay. In order to overcome this partial limitation a CID that would selectively bind to E. coli DHFR and not to endogenous yeast DHFR was envisaged. As an alternative to Mtx, the DHFR inhibitor trimethoprim (TMP) was chosen, which is known for its selectivity for bacterial forms of DHFR [Baccanari, 1982]. Studies have confirmed that while Mtx inhibits growth of wild type Saccrharomyces cerevisiae, TMP does not [Game, 1975], suggesting that TMP could be a superior CID in yeast. Herein is disclosed the design, synthesis, and in vivo activity of Dex-TMP in the yeast three-hybrid assay.

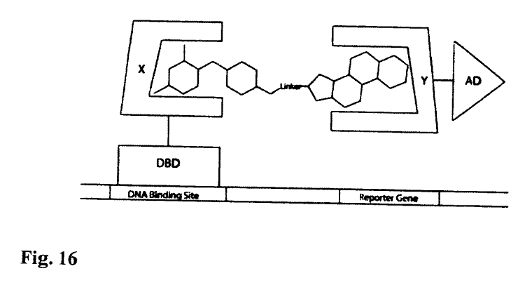

By analogy to our dexamethasone-methotrexate system, a heterodimeric CID was built using the ligand-receptor pairs dexamethasone (Dex)-rat glucocorticoid receptor (GR) and trimethoprim (TMP)-E. coli dihydrofolate reductase (DHFR). Both Dex and TMP can be modified without disrupting receptor binding, making them suitable CIDs [Roth, 1981; Manz, 1983; Govinda, 1980]. Both ligands are cell-permeable and commercially available. The ligand-receptor pair Dex-GR has a K_{D} of 5 nM and has been used successfully in yeast three-hybrid systems [Lin, 2000]. E. Coli dihydrofolate reductase (DHFR) has a K_{I} of 1.3 nM for inhibition by TMP [Baccanari, 1982]. It was hoped that the two interactions would be sufficiently strong to induce protein dimerization and transcription activation in the yeast three-hybrid assay (see FIG. 16).

### Design & Synthesis of Dex-TMP

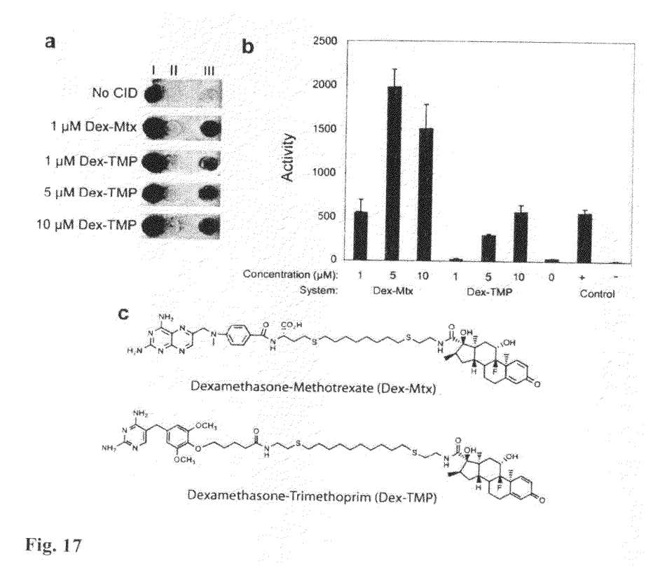

The design and synthesis of the Dex-TMP heterodimer is based on previous syntheses of Dex and TMP derivatives [Roth, 1981; Manz, 1983; Govindan, 1980], with a linker analogous to that for the Dex-Mtx heterodimers most active in the yeast three-hybrid assay (FIG. 17C). Both ligands were coupled as their thiol derivatives to a di-iodo linker. First, Dex was converted to the carboxylic acid by oxidative cleavage using periodate and then derivatized with cystamine using standard peptide coupling reagents. The 4′-methoxy group of TMP was selectively cleaved in 48% hydrobromic acid. The resulting phenol was then derivatized with ethyl 5-bromovalerate using potassium tertbutoxide and converted to the corresponding carboxylic acid. Following saponification 2 equivalents of the TMP acid were reacted with cystamine dihydrochloride under standard peptide coupling conditions to generate the TMP disulfide. Finally, the Dex and TMP disulfides were reduced to their corresponding thiols using tri-n-butylphosphine. The thiol derivatives of the two ligands were coupled to the di-iodo linker sequentially, as for the previous Dex-Mtx CIDs. Thus, the Dex-TMP heterodimer was prepared from two components in a 10-step synthesis in 0.8% overall yield.

### In Vivo Activity in the Yeast Three-Hybrid Assay

Dex-TMP was evaluated for its ability to activate transcription in the yeast three-hybrid assay, using a LexA DNA-binding domain-DHFR protein chimera (LexA-DHFR) and a B42 transcription activation domain-GR protein chimera (B42-GR) and a lacZ reporter gene under control of four tandem LexA operators (FIG. 16). Using standard lacZ transcription assays in both solid and liquid cultures [Adams, 1998], it was demonstrated that Dex-TMP can activate lacZ transcription in the yeast three-hybrid assay (FIGS. 17A&B). X-gal plate assays were carried out as previously reported [Abida, 2002]. Yeast strains were grown on synthetic complete media lacking histidine, uracil, and tryptophan and containing no glucose, 2% galactose, 2% raffinose, and were grown with or without small molecule. In order to determine how effectively Dex-TMP activates transcription in comparison to our previous Dex-Mtx system, the two CIDs were tested side-by-side at concentrations ranging from 1-10 μM in the external growth media. Control experiments established that transcription activation was small molecule dependent, and only background levels of lacZ transcription were detected when both Dex-Mtx and Dex-TMP were omitted. Activation over background levels was observed for both small molecules and at all concentrations, except for 1 μM Dex-TMP. The maximal levels of transcription were observed with 5 μM Dex-Mtx and 10 μM Dex-TMP, respectively. In addition, all three yeast strains were grown in liquid culture and assayed for β-galactosidase activity with ONPG as the substrate. In the liquid culture assays, growth media containing 1, 5, or 10 μM Dex-Mtx resulted in a 22, 79, and 61-fold activation over background levels, respectively. The addition of 5 and 10 μM Dex-TMP resulted in a 12 and 23-fold increase in activation, respectively.

### Results

The new CID Dex-TMP can successfully dimerize the two halves of the transcriptional activator in vivo in the yeast three-hybrid assay, activating transcription of a lacZ reporter gene as shown using β-galactosidase activity assays. Thus, the ligand receptor pair TMP-E. coli DHFR provides a new CID for use in the three-hybrid assay as well as in other in vivo applications of CIDs. This pair may prove particularly useful for applications in mammalian cell lines or even animal studies, when the toxicity of Mtx may prove problematic. However, somewhat surprisingly, the new CID does not induce transcription activation as efficiently as Dex-Mtx. There is evidence, however, that TMP-SLF activates transcription in a yeast three-hybrid assay slightly better than Mtx-SLF (unpublished data). These results point to the complexities of manipulating molecules at the cellular level. There are several plausible explanations for the difference in activity between Dex-TMP and Dex-Mtx. One possible reason for the disparity in activity is the large difference in affinities of the two for DHFR. Mtx binds E. coli DHFR with picomolar affinity (K_{D}=ca. 10 pM) [Appleman, 1998], whereas TMP's affinity is much lower (K_{I}=1.3 nM) [14]. Also, studies have found that although yeast DHFR is not a target of TMP, the small molecule may bind to another yeast protein of unknown function [Barclay, 1993]. Dex-Mtx may have more favorable cell permeability properties than Dex-TMP. In summary, this study provides a new CID pair, TMP-DHFR, which is particularly advantageous for applications in mammalian cell lines and animal studies.

### REFERENCES

Abida et al., (2002) Receptor-dependence of the transcription read-out in a small-molecule three-hybrid system, Chem Biochem. 3, 887.
J. Alam and Cook, J. L., (1990) Anal. Biochem. 188: 245-254.
Althoff, E. A., and Cornish, V. W. (2002), A bacterial small molecule three-hybrid system, Ange. Chem., Int. Ed. Engl. 41, 2327.
Amara et al. (1997), P.N.A.S, USA, 94, 10618-10623.
Baccanari, D. P., Daluge, S. & King, R. W., (1982), Biochemistry 21, 5068 5075.
Baum E Z, Bebernitz G A, Gluzman Y. (1990), Proc Natl Acad Sci USA., 87(24):10023-7.
S. Benkovic, C. Fierke, A. Naylor (1988), Science, 239, 1105-1110.
Bergmann et al. (1994), J. Steroid Biochem. Molec. Biol., 49: 13952.
J. Bolin; Filman, D.; Matthews, D.; Hamlin, R.; Kraut, J. (1982), J. Biol. Chem., 257, 13663-13672.
Brossi et al. (1971), J. Med. Chem. 14:58-59.
Bzik et al., (1987), Proc. Natl. Acad. Sci. U.S.A. 84(23):8360-8364.
Chakraborti, P.; Garabedian, M.; Yamamoto, K.; SS Simons, J. (1991), J. Biol. Chem., 266, 22075-22078.
Chang et al., (1978), Nature 275: 617-624.
T. Clackson (1997) Curr. Opin. Chem. Biol. 1, 210-218.
Clackson, (1998) Curr. Opin. Struct. Biol. 8:451-458.
Clackson et al., (1998), Redesigning an FKBP-ligand interface to generate chemical dimerizers with novel specificity, Proc. Natl. Acad. Sci. U.S.A. 95, 10437-10442.
Coleman, R.; Danishefsky, S. (1989), 28, 157-161.
Crabtree, G.; Schreiber, S. (1996), Trends Biochem. Sci., 21, 418-422.
Edman et al., (1989), Proc. Natl. Acad. Sci. U.S.A. 86(22):8625-8629.
M. Farrar, J. Alberola-Ila, R. Perlmutter (1996), Nature, 383, 178-181.
Fields, S.; Song, O. (1989), Nature, 340, 245-246.
Fling and Richards (1983), Nucl. Acids Res. 11:5147 5158.
Gorman et al. (1982), Mol. Cell Biol. 2: 1044-1051.
Govindan, M.; Manz, B. (1980), Eur. J. Biochem., 108, 47-53.
Gyuris, J.; Golemis, E.; Chertkov, H.; Brent, R. (1993), Cell, 75, 791-803.
Hao et al. (1994), J. Biol. Chem. 269(21):15179-85.
B. Hart, W. Haile, N. Licato, W. Bolanowska, J. McGuire, J. Coward, (1996), J. Med. Chem., 39, 56-65.
Hawkins C J, Wang S L, Hay B A. (1999), Proc. Natl. Acad Sci USA, 96(6):2885-90.
D. Holt, J. Luengo, D. Yamashita, H. Oh, A. Konialan, H. Yen, L. Rozamus, M. Brandt, M. Bossard, M. Levy, D. Eggleston, J. Liang, L. Schultz, T. Stout, J. Clardy, (1993), J. Am. Chem. Soc., 115, 9925-9938.
House, (1972), Modern Synthetic Reactions, publisher Benjamin Cummings.
Hu, J.; O'Shea, E.; Kim, P.; Sauer, R. (1990), Science, 250, 1400-1403.
Hu, J. (1995), Structure, 3, 431-433.
Iversen et al. (1984), J. Urol., Vol. 132, pp. 362-364.
Johnsson, N.; Varshavsky, A. (1994), Proc. Natl. Acad. Sci. USA, 91, 10340-10344.
Kamada S, Kusano H, Fujita H, Ohtsu M, Koya R C, Kuzumaki N, Tsujimoto Y. (1989), Proc Natl Acad Sci USA., 95(15):8532-7.
Karimova, G.; Pidoux, J.; Ullmann, A.; Ladant, D. (1998), Proc. Natl. Acad. Sci. USA, 95, 5752-5756.
Kathryn et al. J. Steroid Biochem. Molec. Biol., 49: 139152.
T. Keenan, D. R. Yaeger, N. L. Courage, C. T. Rollins, M. E.
Pavone, V. M. Rivera, W. Yang, T. Guo, J. F. Amara, T.
Clackson, M. Gilman, D. A. Holt, (1998), Bioorg. Med. Chem., 6, 1309-1335.
King et al. (1992), Proc. Natl. Acad. Sci. USA, Vol. 89, pp. 11322-11326.
Kralovec, J.; Spencer, G.; Blair, A.; Mammen, M.; Singh, M.; Ghose, T. (1989), J. Med. Chem., 32, 2426-2431.
Kuyper et al. (1985), J. Med. Chem. 28: 303-311.
Li et al., (2000), J. Mol. Biol. 295(2), pp. 307-323.
Licitra, E.; Liu, (1996), Proc. Natl. Acad. Sci. USA, 93, 12817-12821.
Lin, H., Abida, W., Sauer, R., Cornish, V. (2000), J. Am. Chem. Soc., 122, 4247-4248.
L. Liu, R. Tanke, M. Miller (1986), J. Org. Chem. 51, 5332-5337.
Manz, B.; Heubner, A.; Kohler, I.; Grill, H.-J.; Pollow, K. (1983), Eur. J. Biochem., 131, 333-338.
Miller et al. (2005), In vivo protein labeling with trimethoprim conjugates: a flexible chemical tag. Nature Methods., Vol. 2, pp. 255-257.
Morandi et al., J. Mol. Biol. 156: 583-607.
PCT International Publication No. WO 02/070662
PCT International Publication No. WO 94/18317
PCT International Publication No. WO 95/02684
PCT International Publication No. WO 96/13613
PCT International Publication No. WO 97/41255
PCT International Publication No. WO 96/06097
PCT International Publication No. WO 2004/078594
PCT International Publication No. WO 01/53355
Picard, D.; Yamamoto, K. (1987), EMBO J., 6, 3333-3338.
Pruschy, M.; Spencer, D.; Kapoor, T.; Miyake, H.; Crabtree, G.; Schreiber, S. (1994), Chem. Biol., 1, 163-172.
Reche et al., (1994), Mol. Biochem. Parasitol. 65(2):247-258.
Rossi, R.; Charlton, C.; Blau, H. (1997), Proc. Natl. Acad. Sci., 94, 8405-8410.
N. Rosenthal (1987), Methods Enzymo. 152: 704-720.
Rosowsky et al. (2002), J. Med. Chem., 45(1):233-241.
Roos, (1993), J. Biol. Chem., 268: 6269-6280.
Roth, B, et al. (1980), J. Med. Chem., 23, p. 535.
Roth, B. et al. (1981), 5. 3′,5′-Dimethoxy-4′-substituted-benzyl analogues of trimethoprim. J. Med. Chem., 24, 933-941.
J. Sambrook, E. Fritsch, T. Maniatis, Molecular Cloning a Laboratory Manual, 2nd ed., Cold Spring Harbor Laboratory Press, Plainview, N.Y., 1989.
A. Shiau and Smith, J. M. (1988), Gene 67: 295-299. Smith T A, Kohorn B D. (1991), Proc Natl Acad Sci USA., 88(12):5159-62.
Spencer et al. (1996), Curr Biol., 6(7): 839-47.
Spencer et al. (1995), Proc Natl Acad Sci USA 92(21): 9805-9.
Spencer, D.; Wandless, T.; Schreiber, S.; Crabtree, G. (1993), Science, 262, 1019-1024.
Then et al. (1982), Rev. Infect. Dis., Vol. 4, pp. 372-377.
U.S. Pat. No. 3,049,544, Stenbuck et al.
U.S. Pat. No. 3,341,541, Hoffer et al.
U.S. Pat. No. 5,341,541, Sham.
U.S. Pat. No. 5,468,614, Fields et al.
U.S. Pat. No. 5,503,977, Johnsson et al.
U.S. Pat. No. 5,585,245, Johnsson et al.
U.S. Pat. No. 5,928,868, Liu et al.
U.S. Publication No. 2003-0203471, Althoff et al.
Wagner, J.; Lerner, R.; Barbas, C. (1995), Science, 270, 1797-1800.
Wagner, R.; Rhoades, T.; Or, Y.; Lane, B.; Hsieh, G.; Mollison, K.; Luly, J. (1998), J. Med. Chem., 41, 1764-1776.
Walzer et al. (1993), Anti. Agents and Chem. Vol. 37, pp. 1436-1443.
Wüst and Schwarzenbach (1983), Anti. Agents and Chem. Vol. 23, pp. 490-492.
Yang et al. (1995), Nucleic Acid Research, 23, 1152-1156.
Zwyno-vanGinkel, (1997), FEMS Microbiol. Letts., 156:69-78.
Lin, H. N., H. Y. Tao, and V. W. Cornish, Directed evolution of a glycosynthase via chemical complementation. Journal of the American Chemical Society, 2004. 126(46): p. 15051-15059.
Lin, H. N. and V. W. Cornish, In vivo protein-protein interaction assays: Beyond proteins. Angewandte Chemie-International Edition, 2001. 40(5): p. 871-875.
Johnsson, N. and K. Johnsson, A fusion of disciplines: Chemical approaches to exploit fusion proteins for functional genomics. Chembiochem, 2003. 4(9): p. 803-810.
Becker, F., et al., A three-hybrid approach to scanning the proteome for targets of small molecule kinase inhibitors. Chemistry & Biology, 2004. 11(2): p. 211-223.
Mapp, A. K., Regulating transcription: a chemical perspective. Organic & Biomolecular Chemistry, 2003. 1(13): p. 2217-2220.
Hu, J. C., Model systems: Studying molecular recognition using bacterial n-hybrid systems. Trends in Microbiology, 2001. 9(5): p. 219-222.
Spencer, D. M., et al., Controlling Signal-Transduction with Synthetic Ligands. Science, 1993. 262(5136): p. 1019-1024.
Hussey, S. L., S. S. Muddana, and B. R. Peterson, Synthesis of a beta-estradiol-biotin chimera that potently heterodimerizes estrogen receptor and streptavidin proteins in a yeast three-hybrid system. Journal of the American Chemical Society, 2003. 125(13): p. 3692-3693.
Lin, H. N., et al., Dexamethasone-methotrexate: An efficient chemical inducer of protein dimerization in vivo. Journal of the American Chemical Society, 2000. 122(17): p. 4247-4248.
Abida, W. M., et al., Receptor-dependence of the transcription read-out in a small-molecule three-hybrid system. Chembiochem, 2002. 3(9): p. 887-895.
Baker, K., et al., An optimized dexamethasone-methotrexate yeast 3-hybrid system for high-throughput screening of small molecule-protein interactions. Analytical Biochemistry, 2003. 315(1): p. 134-137.
Chakraborti, P. K., et al., Creation of Super Glucocorticoid Receptors by Point Mutations in the Steroid Binding Domain. Journal of Biological Chemistry, 1991. 266(33): p. 22075-22078.
Sasso, S. P., et al., Thermodynamic Study of Dihydrofolate-Reductase Inhibitor Selectivity. Biochimica Et Biophysica Acta-Protein Structure and Molecular Enzymology, 1994. 1207(1): p. 74-79.
Baccanari, D. P., S. Daluge, and R. W. King, Inhibition of Dihydrofolate-Reductase—Effect of Reduced Nicotinamide Adenine-Dinucleotide Phosphate on the Selectivity and Affinity of Diaminobenzylpyrimidines. Biochemistry, 1982. 21(20): p. 5068-5075.
Game, J. C., J. G. Little, and R. H. Haynes, Yeast Mutants Sensitive to Trimethoprim. Mutation Research, 1975. 28(2): p. 175-182.
Roth, B., et al., 2,4-Diamino-5-Benzylpyrimidines and Analogs as Anti-Bacterial Agents .5. 3′,5′-Dimethoxy-4′-Substituted-Benzyl Analogs of Trimethoprim. Journal of Medicinal Chemistry, 1981. 24(8): p. 933-941.
Manz, B., et al., Synthesis of Biotin-Labeled Dexamethasone Derivatives—Novel Hormone-Affinity Probes. European Journal of Biochemistry, 1983. 131(2): p. 333-338.
Govindan, M. V. and B. Manz, 3-Step Purification of Glucocorticoid Receptors from Rat-Liver. European Journal of Biochemistry, 1980. 108(1): p. 47-53.
Brossi, A., et al., Synthesis and Chemotherapeutic Activity of 2 Metabolites of Trimethoprim. Journal of Medicinal Chemistry, 1971. 14(1): p. 58-&.
Adams, A., et al., eds. Methods in Yeast Genetics. 1998, Cold Spring Laboratory Press: Plainview.
Appleman, J. R., et al., Role of Aspartate-27 in the Binding of Methotrexate to Dihydrofolate-Reductase from Escherichia-coli. Journal of Biological Chemistry, 1988. 263(19): p. 9187-9198.
Barclay, B. J., M. G. Nagel, and T. Huang, Dihydrofolate reductase is not the target of trimethoprim in Saccharomyces cerevisiae. Adv. Exp. Med. Biol., 1993. 338: p. 551-4.

1. A method for identifying a molecule as being able to bind a protein target in a cell, comprising:
(a) covalently bonding the molecule to trimethoprim (TMP) to form a screening molecule;
(b) introducing the screening molecule into the cell which (A) expresses (i) a first fusion protein comprising a binding domain capable of binding TMP, and (ii) a second fusion protein comprising the protein target, and (B) comprises a reporter gene, wherein one of the fusion proteins comprises a DNA-binding domain and the other fusion protein comprises a transcription activation domain and wherein expression of the reporter gene is conditioned on the proximity of the DNA-binding domain to the transcription activation domain; and
(c) determining whether the screening molecule binds to the first fusion protein and to the second fusion protein by determining whether the cell expresses the reporter gene,
wherein expression of the reporter gene indicates that the molecule binds the protein target and wherein lack of expression of the reporter gene indicates that the molecule does not bind the protein target.

2. (canceled)

3. A method for identifying a protein target as capable of binding a molecule in a cell, comprising:
(a) providing a screening molecule comprising trimethoprim (TMP) covalently bonded to the molecule;
(b) introducing the screening molecule into the cell which (A) expresses (i) a first fusion protein comprising a binding domain capable of binding TMP, and (ii) a second fusion protein comprising the target protein, and (B) comprises a reporter gene, wherein one of the fusion proteins comprises a DNA-binding domain and the other fusion protein comprises a transcription activation domain and wherein expression of the reporter gene is conditioned on the proximity of the DNA-binding domain to the transcription activation domain; and
(c) determining whether the protein target is capable of binding the molecule by determining whether the cell expresses the reporter gene,
wherein expression of the reporter gene indicates that the protein target is capable of binding the molecule and wherein lack of expression of the reporter gene indicates that the protein target is not capable of binding the molecule.

4. (canceled)

5. (canceled)

6. The method of claim 1 or 3, where the cell is a yeast cell, a bacteria cell, an insect cell or a mammalian cell.

7. (canceled)

8. The method of claim 1 or 3, wherein the first fusion protein or the second fusion protein is dihydrofolate reductase-LexA.

9. The method of claim 1 or 3, wherein the first fusion protein or the second fusion protein is dihydrofolate reductase-B42.

10. (canceled)

11. (canceled)

12. The method of claim 1 or 3 wherein the reporter gene is lacZ, leu2, ura3, his3, or trp, trp1, trp2, ade2, lys2, cyh1, can1, gfp, cat, araBAD, aaDa, KanR, or Cm.

13. The method of claim 1 or 3, wherein the transcription activation domain is αNTD.

14. The method of claim 1 or 3, wherein the DNA-binding domain is λcI, AraC, LexA, Gal4, or zinc fingers.

15. The method of claim 1 or 3, wherein TMP has the following structure:

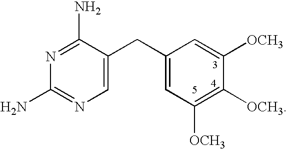

16. (canceled)

17. (canceled)

18. (canceled)

19. The method of claim 1 or 3, wherein TMP has the following structure: 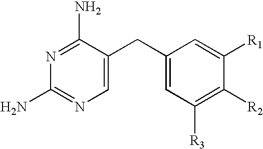
wherein if TMP is bonded to the screening molecule at R₁, then R₂ may be —H, —F, —Cl, —OH, —I, —Br, —O(CH₂)₇CH₃, —CH₂OH, —O(CH₂)₆CH₃, —OCH₂CH₂OCH₃, —CH₂O(CH₂)₃CH₃, —OCH₂CONH₂, —CH₂OCH₃, —CH₃, —O(CH₂)₃CH₃, —O(CH₂)₅CH₃, —OCH₃, —NO₂, —OSO₂CH₃, —OCH₂C₆H₅, —CF₃, —NH₂, —OCF₃, —N(CH₃)₂, —NHCOCH₃, —NHCOCH₃, —O(CH₂)₂OCH₃, and —HCH₂CH₂OCH₃; and R₃ may be —H, —OH, —CH₂OH, and —OCH₃;
wherein if TMP is bonded to the screening molecule at R₂, then R₁ may be —H, —F, —Cl, —OH, —I, —Br, —O(CH₂)₇CH₃, —CH₂OH, —O(CH₂)₆CH₃, —OCH₂CH₂OCH₃, —CH₂O(CH₂)₃CH₃, —OCH₂CONH₂, —CH₂OCH₃, —CH₃, —O(CH₂)₃CH₃, —O(CH₂)₅CH₃, —OCH₃, —NO₂, —OSO₂CH₃, —OCH₂C₆H₅, —CF₃, —NH₂, —OCF₃, —N(CH₃)₂, —NHCOCH₃, —NHCOCH₃, —O(CH₂)₂OCH₃, and —HCH₂CH₂OCH₃; and R₃ may be —H, —OH, —CH₂OH, and —OCH₃;
wherein if TMP is bonded to the screening molecule at R₃, then R₁ may be —H, —F, —Cl, —OH, —I, —Br, —O(CH₂)₇CH₃, —CH₂OH, —O(CH₂)₆CH₃, —OCH₂CH₂OCH₃, —CH₂O(CH₂)₃CH₃, —OCH₂CONH₂, —CH₂OCH₃, —CH₃, —O(CH₂)₃CH₃, —O(CH₂)₅CH₃, —OCH₃, —NO₂, —OSO₂CH₃, —OCH₂C₆H₅, —CF₃, —NH₂, —OCF₃, —N(CH₃)₂, —NHCOCH₃, —NHCOCH₃, —O(CH₂)₂OCH₃, and —HCH₂CH₂OCH₃; and R₂ may be —H, —F, —Cl, —OH, —I, —Br, —O(CH₂)₇CH₃, —CH₂OH, —O(CH₂)₆CH₃, —OCH₂CH₂OCH₃, —CH₂O(CH₂)₃CH₃, —OCH₂CONH₂, —CH₂OCH₃, —CH₃, —O(CH₂)₃CH₃, —O(CH₂)₅CH₃, —OCH₃, —NO₂, —OSO₂CH₃, —OCH₂C₆H₅, —CF₃, —NH₂, —OCF₃, —N(CH₃)₂, —NHCOCH₃, —NHCOCH₃, —O(CH₂)₂OCH₃, and —HCH₂CH₂OCH₃.

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. A method for identifying a first molecule as a competitive inhibitor of a second molecule's binding to a protein target in a cell, comprising:
(a) covalently bonding the second molecule to trimethoprim (TMP) to form a screening molecule;
(b) introducing the screening molecule into the cell which (A) expresses (i) a first fusion protein comprising a binding domain capable of binding TMP, and (ii) a second fusion protein comprising the protein target, and (B) comprises a reporter gene, wherein one of the fusion proteins comprises a DNA-binding domain and the other fusion protein comprises a transcription activation domain and wherein expression of the reporter gene is conditioned on the proximity of the DNA-binding domain to the transcription activation domain;
(c) quantitating cellular expression of the reporter gene;
(d) introducing the first molecule into the cell repeating steps (b) and (c) in the presence of the first molecule;
wherein a decreased expression of the reporter gene in the presence of the first molecule indicates that the first molecule is a competitive inhibitor of the second molecule's binding to the protein target.

40. (canceled)

41. (canceled)

42. (canceled)

43. (canceled)

44. (canceled)

45. (canceled)

46. (canceled)

47. (canceled)

48. (canceled)

49. (canceled)

50. The method of claim 39, wherein TMP has the following structure:

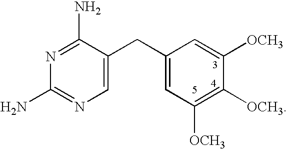

51. (canceled)

52. (canceled)

53. (canceled)

54. The method of claim 39, wherein TMP has the following structure: 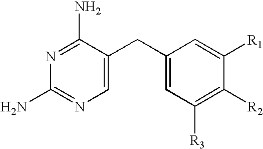
wherein if TMP is bonded to the screening molecule at R₁, then R₂ may be —H, —F, —Cl, —OH, —I, —Br, —O(CH₂)₇CH₃, —CH₂OH, —O(CH₂)₆CH₃, —OCH₂CH₂OCH₃, —CH₂O(CH₂)₃CH₃, —OCH₂CONH₂, —CH₂OCH₃, —CH₃, —O(CH₂)₃CH₃, —O(CH₂)₅CH₃, —OCH₃, —NO₂, —OSO₂CH₃, —OCH₂C₆H₅, —CF₃, —NH₂, —OCF₃, —N(CH₃)₂, —NHCOCH₃, —NHCOCH₃, —O(CH₂)₂OCH₃, and —HCH₂CH₂OCH₃; and R₃ may be —H, —OH, —CH₂OH, and —OCH₃;
wherein if TMP is bonded to the screening molecule at R₂, then R₁ may be —H, —F, —Cl, —OH, —I, —Br, —O(CH₂)₇CH₃, —CH₂OH, —O(CH₂)₆CH₃, —OCH₂CH₂OCH₃, —CH₂O(CH₂)₃CH₃, —OCH₂CONH₂, —CH₂OCH₃, —CH₃, —O(CH₂)₃CH₃, —O(CH₂)₅CH₃, —OCH₃, —NO₂, —OSO₂CH₃, —OCH₂C₆H₅, —CF₃, —NH₂, —OCF₃, —N(CH₃)₂, —NHCOCH₃, —NHCOCH₃, —O(CH₂)₂OCH₃, and —HCH₂CH₂OCH₃; and R₃ may be —H, —OH, —CH₂OH, and —OCH₃;
wherein if TMP is bonded to the screening molecule at R₃, then R₁ may be —H, —F, —Cl, —OH, —I, —Br, —O(CH₂)₇CH₃, —CH₂OH, —O(CH₂)₆CH₃, —OCH₂CH₂OCH₃, —CH₂O(CH₂)₃CH₃, —OCH₂CONH₂, —CH₂OCH₃, —CH₃, —O(CH₂)₃CH₃, —O(CH₂)₅CH₃, —OCH₃, —NO₂, —OSO₂CH₃, —OCH₂C₆H₅, —CF₃, —NH₂, —OCF₃, —N(CH₃)₂, —NHCOCH₃, —NHCOCH₃, —O(CH₂)₂OCH₃, and —HCH₂CH₂OCH₃; and R₂ may be —H, —F, —Cl, —OH, —I, —Br, —O(CH₂)₇CH₃, —CH₂OH, —O(CH₂)₆CH₃, —OCH₂CH₂OCH₃, —CH₂O(CH₂)₃CH₃, —OCH₂CONH₂, —CH₂OCH₃, —CH₃, —O(CH₂)₃CH₃, —O(CH₂)₅CH₃, —OCH₃, —NO₂, —OSO₂CH₃, —OCH₂C₆H₅, —CF₃, —NH₂, —OCF₃, —N(CH₃)₂, —NHCOCH₃, —NHCOCH₃, —O(CH₂)₂OCH₃, and —HCH₂CH₂OCH₃.
